SEQUENCE LISTING (37 sequences)

SEQ ID NO: 1 - protein, 305 aa, synthetic construct
  source 1..305: note = H2B10
SKRGIDVSEWQGDIDFNAVKASGVEFVIIRAGYGIGCKDKWFEQNYRKAKTCGLDVGAYWYSYANSGFEAAEEAQSCVNMLSGKSFEYPVYFDLEEKSQLNRGRAFCDSLITGFCNKLESCGYYAGFYTSLSTANNLVSAHVRNRYALWIAQWNTHCSYQGSYGLWQYSSSGSVPGVAGRVDMDYAYVDYPSIIKNVGLNGYKNGGSYTAPQISSIDEVAREVINGDWGNGNERKQRLTSAGYDYASVQNKVNELLGVKAYRKSVDELAREVIRGTWGNGSTRKQRLTQAGYDYNAVQKRVNELL

SEQ ID NO: 2 - protein, 196 aa, synthetic construct
  source 1..196: note = H1
MSKKGIDVSEWQGDIDFNAVKASGVEFVIIRAGYGIGCKDKWFEQNYRKAKTAGLDVGAYWYSYANSGFEAAEEAQSLMNMLSGKSFEYPVYFDLEEKSQLNRGRAFCDSLITSFCNKLEACGYYAGFYTSLSTANNLVSAHVRNRYALWIAQWNTHCNYQGSYGLWQYSSNGSVPGVAGRVDMDYAYVDYPSIIK

SEQ ID NO: 3 - protein, 196 aa, synthetic construct
  source 1..196: note = H2
MSKRGIDVSEWQGDIDFNAVKASGVEFVIIRAGYGIGCKDKWFEQNYRKAKTCGLDVGAYWYSYANSGFEAAEEAQSCVNMLSGKSFEYPVYFDLEEKSQLNRGRAFCDSLITGFCNKLESCGYYAGFYTSLSTANNLVSAHVRNRYALWIAQWNTHCSYQGSYGLWQYSSSGSVPGVAGRVDMDYAYVDYPSIIK

SEQ ID NO: 4 - protein, 196 aa, synthetic construct
  source 1..196: note = H3
MSKKGIDVSVWQGDIDFNSVKASGVEFVIIRAGYGIGHKDKWFEENYRKAKTAGLDVGSYWYSYASSAGEASEEAQSCVNILSGKSFEYPIYFDLEEKSQLNRGRDFCDSLITSFCNKLEACGYYAGFYTSLSVANNLVSSHVRDRYALWIAQWNTHCSYQGSYGLWQYSSSGSVNGIAGRVDMDYAYVDYPSVIK

SEQ ID NO: 5 - protein, 196 aa, synthetic construct
  source 1..196: note = H4
MSKKGIDVSEWQGDIDFNAVKASGVEFVIIRAGYGIGCKDKWFEQNYRKAKTCGLDVGAYWYSYANSGFEAAEEAQSCVNMLSGKSFEYPVYFDLEEKSQLNRGRAFCDSLITSFCSKLETYGYYAGFYTSLSVVNNLVSAHVRDRYALWIAQWNTHCSYQGSYGLWQYSSSGSVPGVAGRVDMDYAYVDYPSIIK

SEQ ID NO: 6 - protein, 196 aa, synthetic construct
  source 1..196: note = H5
MSKKGIDVSEWQGDIDFNAVKASGVEFVIIRAGYGIGCKDKWFEQNYRKAKTAGLDVGAYWYSYANSSSEAAEEAQSCANMLSGKSFEYPVYFDLEEKSQLNRGRAFCDSLITGFCSKLEACGYYAGFYTSLSTANNLVSAHVRNRYALWIAQWNTHCSYQGSYGLWQYSSNGSVPGVAGRVDMDYAYKDYPSIIK

SEQ ID NO: 7 - protein, 196 aa, synthetic construct
  source 1..196: note = H7
MSKKGIDVSEWQGDIDFNAVKASGVEFVIIRAGYGIGCKDKWFEQNYRKAKTAGLDVGAYWYSYANSASEAAEEAQSCANMLSGKSFEYPVYFDLEEKSQLNRGRAFCDSLITSFCSKLETYGYYAGFYTSLSTANNLVSSHVRNRYALWIAQWNTHCSYQGSYGLWQYSSSGSVPGVAGRVDMDYAYKDYPSIIK

SEQ ID NO: 8 - protein, 196 aa, synthetic construct
  source 1..196: note = H10
MSKRGIDVSVWQGDIDFNAVKASGVEFVIIRAGYGIGHKDKWFEENYRKAKTVGLDVGAYWYSYASSAGEASEEAQSCVNILSGKSFEYPVYFDLEEKSQLNRGRDFCDSLITSFCNKLEACGYYAGFYTSLSVANNLVSSHVRDRYALWIAQWNTHCSYQGSYGLWQYSSSGSVNGIAGRVDMDYAYVDYPSVIK

SEQ ID NO: 9 - protein, 196 aa, synthetic construct
  source 1..196: note = H11
MSKRGIDVSVWQGDIDFNAVKASGVEFVIIRAGYGIGHKDKWFEQNYRKAKTTGLDVGAYWYSYASSAGEAAEEAQSCVNILSGKSFEYPVYFDLEEKSQLNRGRDFCDSLITSFCNKLETYGYYAGFYTSLSVANNLVSSHVRDRYALWIAQWNTHCDYQGSYGLWQYSSSGSVDGIAGRVDMDYTYVDYPSVIK

SEQ ID NO: 10 - protein, 196 aa, synthetic construct
  source 1..196: note = H12
MSKKGIDVSVWQGDIDFNAVKASGVEFVIIRAGYGIGHKDKWFEENYRKAKTAGLDVGSYWYSYASSAGEVALEAQSCVNILSGKSFEYPVYFDLEEKSQLNRGRDFCDSLITSFCNKLEACGYYAGFYTSLSVANNLVSSHVRDRYALWIAQWNTHCSYQGSYGLWQYSSSGSVNGIAGRVDMDYAYVDYPSVIK

SEQ ID NO: 11 - protein, 49 aa, synthetic construct
  source 1..49: note = B1_N
DQAARTSSIDEVAREVINGAWGNGSTRKQRLTSAGYDYASVQNKVNELL

SEQ ID NO: 12 - protein, 49 aa, synthetic construct
  source 1..49: note = B1_C
GVKACRKSVDELAREVIRGAWGNGSTRKQRLAQAGYDYDTVQKRVNELL

SEQ ID NO: 13 - protein, 49 aa, synthetic construct
  source 1..49: note = B2_N
DQAARTSSIDEVAREVINGAWGNGNERKQRLTSAGYDYASVQNKVNELL

SEQ ID NO: 14 - protein, 49 aa, synthetic construct
  source 1..49: note = B2_C
GVKACRKSVDEIAREVIRGTWGNGSTRKQRLTQAGYDYDTVQKRVNELL

SEQ ID NO: 15 - protein, 49 aa, synthetic construct
  source 1..49: note = B3_N
YTAPQTSSIDEVAREVINGDWGNGNDRKNRLISAGYDYASVQNKVNELL

SEQ ID NO: 16 - protein, 49 aa, synthetic construct
  source 1..49: note = B3_C
GVKAYRKSVDELAREVIRGTWGNGSMRKHRLTQAGYDYDAVQKRVNELL

SEQ ID NO: 17 - protein, 49 aa, synthetic construct
  source 1..49: note = B4_N
DQATRTSSIDEVAREVINGAWGNGNERKQRLTSAGYDYASVQNKVNKLL

SEQ ID NO: 18 - protein, 49 aa, synthetic construct
  source 1..49: note = B4_C
GVKAYRKSVDELAREVIRGTWGNGNERKQRLAQAGYDYDTVQKRVNELL

SEQ ID NO: 19 - protein, 49 aa, synthetic construct
  source 1..49: note = B5_N
DQAARTSSIDEVAREVINGAWGNGNERKQRLTQAGYDYTSVQNKVNKLL

SEQ ID NO: 20 - protein, 49 aa, synthetic construct
  source 1..49: note = B5_C
GVKACRKSVDELAREVIRGTWGNGNERKNRLTQAGYDYDTVQKRVNELL

SEQ ID NO: 21 - protein, 49 aa, synthetic construct; identical to SEQ ID NO: 19
  source 1..49: note = B7_N

SEQ ID NO: 22 - protein, 49 aa, synthetic construct; identical to SEQ ID NO: 20
  source 1..49: note = B7_C

SEQ ID NO: 23 - protein, 49 aa, synthetic construct
  source 1..49: note = B10_N
YTAPQISSIDEVAREVINGDWGNGNERKQRLTSAGYDYASVQNKVNELL

SEQ ID NO: 24 - protein, 49 aa, synthetic construct
  source 1..49: note = B10_C
GVKAYRKSVDELAREVIRGTWGNGSTRKQRLTQAGYDYNAVQKRVNELL

SEQ ID NO: 25 - protein, 49 aa, synthetic construct
  source 1..49: note = B11_N
YTAPQTSSIDEVAREVINGDWGNGNERKNRLTSAGYDYTSVQNKVNELL

SEQ ID NO: 26 - protein, 49 aa, synthetic construct
  source 1..49: note = B11_C
GVKAYRKSVDELAREVIRGTWGNGSTRKQRLTQAGYDYDAVQKRVNELL

SEQ ID NO: 27 - protein, 49 aa, synthetic construct
  source 1..49: note = B12_N
YTAPQTSSIDEVAREVINGDWGNGIERKNRLTSAGYDYTSVQNKVNELL

SEQ ID NO: 28 - protein, 49 aa, synthetic construct
  source 1..49: note = B12_C
GVKAYRKSVDELAREVIRGTWGNGKTRKQRLTQAGYDYNAVQKRVNELL

SEQ ID NO: 29 - protein, 12 aa, synthetic construct
  source 1..12: note = L1
NAGLNGCKNGGS

SEQ ID NO: 30 - protein, 12 aa, synthetic construct
  source 1..12: note = L2
NVGLNGCKNGGS

SEQ ID NO: 31 - protein, 12 aa, synthetic construct
  source 1..12: note = L3, L12
NAGLNGYQNGGS

SEQ ID NO: 32 - protein, 12 aa, synthetic construct
  source 1..12: note = L4
NVGLNGCKNGGS

SEQ ID NO: 33 - protein, 12 aa, synthetic construct
  source 1..12: note = L5, L6, L7
NAGLNGCKNGGS

SEQ ID NO: 34 - protein, 12 aa, synthetic construct
  source 1..12: note = L10
NAGLNGYKNGGS

SEQ ID NO: 35 - protein, 12 aa, synthetic construct
  source 1..12: note = L11
KAGLNGYKNGGS

SEQ ID NO: 36 - protein, 12 aa, synthetic construct
  source 1..12: note = Lx
  VARIANT 1: note = X is N or K
  VARIANT 2: note = X is A or V
  VARIANT 7: note = X is Y or C
  VARIANT 8: note = X is K or Q
XXGLNGXXNGGS

SEQ ID NO: 37 - protein, 306 aa, synthetic construct
  source 1..306: note = H2B10B11
MSKRGIDVSEWQGDIDFNAVKASGVEFVIIRAGYGIGCKDKWFEQNYRKAKTCGLDVGAYWYSYANSGFEAAEEAQSCVNMLSGKSFEYPVYFDLEEKSQLNRGRAFCDSLITGFCNKLESCGYYAGFYTSLSTANNLVSAHVRNRYALWIAQWNTHCSYQGSYGLWQYSSSGSVPGVAGRVDMDYAYVDYPSIIKNVGLNGYKNGGSYTAPQISSIDEVAREVINGDWGNGNERKQRLTSAGYDYASVQNKVNELLGVKAYRKSVDELAREVIRGTWGNGSTRKQRLTQAGYDYDAVQKRVNELL

(19) United States
(12) Patent Application Publication
(10) Pub. No.: US 2026/0306591 A1
(43) Pub. Date: Oct. 8, 2026

(54) INTRAVAGINAL FORMULATIONS OF GARDNERELLA ENDOLYSINS

(71) Applicant: BioNTech SE, Mainz (DE)

(72) Inventors: Ann-Katrin Kieninger, Wien (AT); Lorenzo Corsini, Wien (AT); Lenka Podpera Tisakova, Wien (AT); Timo Schwebs, Wien (AT); Maria Protano, Wien (AT); Albina Poljak, Wien (AT)

(21) Appl. No.: 19/482,194

(22) PCT Filed: May 8, 2024
(86) PCT No.: PCT/EP2024/062837

§ 371 Date: Nov. 6, 2025

(30) Foreign Application Priority Data
May 9, 2023 (EP) 23172423.8
Dec. 12, 2023 (EP) 23215987.1

(51) Int. Cl. A61K 38/47; A61K 47/02; A61K 47/32; A61P 31/04

(52) U.S. Cl. CPC A61K 38/47; A61K 47/02; A61K 47/32; A61P 31/04

(57) ABSTRACT

The present invention relates to new pharmaceutical compositions comprising an effective amount of an active agent and a pharmaceutical acceptable excipient, wherein said active agent is a polypeptide having a killing activity against Gardnerella and wherein said excipient is a polyacrylic acid polymer. The present invention further relates to uses of the pharmaceutical compositions in the treatment of Gardnerella infections, in particular bacterial vaginosis.

### CROSS-REFERENCE TO RELATED APPLICATIONS

This application is a United States National Phase Application filed under 35 U.S.C. § 371 of International Patent Application No. PCT/EP2024/062837, filed May 8, 2024, which claims priority to European Patent Application No. 23172423.8, filed May 9, 2023, and European Patent Application No. 23215987.1, filed Dec. 12, 2023. The contents of each of the foregoing applications are incorporated herein by reference in their entireties.

### INCORPORATION-BY-REFERENCE OF SEQUENCE LISTING

The instant application contains a Sequence Listing which has been submitted electronically in XML format via Patent Center and is hereby incorporated by reference in its entirety. Said XML copy, created on Nov. 6, 2025, is named “000491-0003-301-SL.xml” and is 43,531 bytes in size.

### TECHNICAL FIELD

The present invention relates to new pharmaceutical compositions for the treatment of infections caused by bacteria of the genus Gardnerella. The pharmaceutical compositions of the invention are characterized in that they comprise a Gardnerella-specific polypeptide as active agent and a polyacrylic acid polymer as excipient. The present invention further relates to medical uses of the pharmaceutical composition of the invention as well as methods of treatment using the same.

### BACKGROUND INFORMATION

Bacterial vaginosis (BV), also been referred to in the literature as bacterial vaginitis, non-specific vaginosis and non-specific vaginitis, is the most common vaginal infection worldwide and is associated with significant adverse consequences including preterm labor and delivery, post-partum enodmetritis and an increased risk of HIV acquisition. It is a dysbiosis of the vagina where the commensal Lactobacilli are displaced by a polymicrobial biofilm, the pH increases from the natural 3.5-4.5 up to 5.5, and a malodorous fluid forms. Reported prevalence rates range from 10-40% depending upon the population studied. However, suboptimal methods of diagnosis and a high percentage of asymptomatic patients make the true prevalence of BV difficult to ascertain. Gardnerella vaginalis (G. vaginalis) is a bacterial species associated with BV.

The etiopathogenesis of BV remains poorly understood. It is most commonly defined as a pathological state characterized by the loss of normal vagina flora, particularly of H₂O₂-producing species of Lactobacillus, and the simultaneous overgrowth of anaerobic bacteria including G. vaginalis, Mobiluncus species, and Mycoplasma hominis. Recent data, however, suggest a primary role for G. vaginalis as a specific and sexually transmitted etiological agent in BV (Muzny et al., 2016, J. of Infect. Dis. 214 Suppl. 1., S1).

In the 1950s, abundant small, pleomorphic gram-variable rods were observed in the genital tract of women with BV. This organism, first called Haemophilus vaginalis and repeatedly renamed as more information about its characteristics became available, is now classified as G. vaginalis which, until 2018, was considered to be the sole member of the genus Gardnerella. However, in early 2019 it was shown that the genus Gardnerella actually contains at least 13 species, and the most frequent ones were renamed G. vaginalis sensu stricto, G. leopoldii, G. piotii, and G. swidsinskii (Vaneechoutte et al., 2019 Int. J. Syst. Evol. Biol. 898661).

Bacteria of the genus Gardnerella are special in that they are Gram-variable, i.e., they do not form the outer membrane defining the Gram-negative species. The cell wall is generally very thin and has only 10% or less content of peptidoglycan, which is why the crystal violet dye used for Gram staining does not always yield the deep purple color typical for Gram-positive species. Rather, Gardnerella cells can appear both Gram positive and negative in a Gram staining. Phylogenetic analysis based on 16S rRNA places Gardnerella in the gram-positive family Bifidobacteriales.

During BV, the epithelial surface is covered with a dense collection of G. vaginalis in a biofilm that is frequently recalcitrant to treatment. Biofilms are adherent communities of microorganisms held together by a polymeric matrix composed of polysaccharides, proteins and/or nucleic acids. The distinct gene expression pattern, as well as the physical structure of biofilms increases bacterial resistance to many negative stimuli including chemical disinfectants, pH extremes, host immune defenses and antibiotics. Standard of BV treatment are the antibiotics Metronidazole and Clindamycin, which however often fail to eradicate the biofilm, so that recurrence rates are up to 60% within 6 months. Furthermore, treatment with antibiotics wipes the vaginal microbiome, despite leaving some rests of viable biofilm, which opens this ecological niche for other pathogens, e.g., fungi. A frequent effect of BV treatments is therefore candidosis. Treatment of BV was also attempted with probiotics, specifically with beneficial Lactobacilli supposed to re-colonize the vagina. However, several clinical trials failed to show a benefit.

Endolysins are promising alternatives to the current antibiotics, due to their ability to eradicate biofilms, their low propensity to the development of resistance, and their specificity to individual genera or species of bacteria. Natural and genetically engineered Gardnerella-specific endolysins have been described (WO 2020/225335 A1, Landlinger et al., (2021, Pathogens 10, 1-19), or WO 2020/229802 A1). WO 2020/225335 describes specific recombinant Gardnerella-specific endolysins (e.g., H₂B10) for use in a method of treating a Gardnerella infection such as BV, wherein said bacterial vaginosis is caused by Gardnerella vaginalis sensu stricto, Gardnerella leopoldii, Gardnerella piotii and/or Gardnerella swidsinskii. Examples 6 and 7 of WO 2020/225335 demonstrate that for example the endolysin H2B10 (as a representative of recombinant Gardnerella-specific endolysins) is superior to the antibiotics Metronidazole and Clindamycin, particularly in terms of minimal inhibitory concentration (MIC) on the growth in suspension of the Gardnerella strains. The results of WO 2020/225335 therefore document that (recombinant) Gardnerella-specific endolysins are superior to antibiotics in the treatment of BV in a general manner. In other words, (recombinant) Gardnerella-specific endolysins have been shown to be generally more effective on the growth in suspension of the Gardnerella strains as can be derived from the generally lower MIC values of the endolysins across the different Gardnerella species in comparison to those of the antibiotics.

Despite these recent advances, there is still a need in the art for compositions and/or formulations that allow an even more effective treatment of infections caused by Gardnerella species. The technical problem underlying the present invention can thus be formulated as the provision of improved pharmaceutical compositions for the treatment of infections caused by bacteria of the genus Gardnerella.

The technical problem is solved by provision of the herein provided embodiments and claims.

### SUMMARY OF THE INVENTION

The present invention relates, inter alia, to the following embodiments:
1. A pharmaceutical composition comprising an effective amount of an active agent and a pharmaceutical acceptable excipient, wherein said active agent is a polypeptide having a killing activity against Gardnerella and wherein said excipient is a polyacrylic acid polymer.
2. The pharmaceutical composition according to embodiment 1, wherein said polypeptide is an endolysin having a killing activity against Gardnerella.
3. The pharmaceutical composition according to embodiment 2, wherein said endolysin comprises or consists of
   (i) a N-terminal catalytic domain, or a functional variant thereof;
   (ii) a C-terminal cell-wall binding region, or a functional variant thereof, wherein the C-terminal cell-wall binding region comprises or consists of at least one cell-wall binding domain; and
   (iii) optionally a linker region between the N-terminal catalytic domain and the C-terminal cell-wall binding region.
4. The pharmaceutical composition according to any one of the preceding embodiments, wherein said polypeptide is a polypeptide having at least 80% sequence identity with the amino acid sequence as provided in SEQ ID NO: 1 or SEQ ID NO:37 and having a killing activity against Gardnerella.
5. The pharmaceutical composition according to any one of the preceding embodiments, wherein said polypeptide is a recombinant endolysin comprising the amino acid sequence provided in SEQ ID NO: 1 or SEQ ID NO:37.
6. The pharmaceutical composition according to any one of the preceding embodiments, wherein said polypeptide has a killing activity against Gardnerella vaginalis sensu stricto, Gardnerella leopoldii, Gardnerella piotii and/or Gardnerella swidsinskii, or any other species in the genus Gardnerella.
7. The pharmaceutical composition according to any one of the preceding embodiments, wherein said polypeptide has a killing activity against Lactobacillus iners.
8. The pharmaceutical composition according to any one of the preceding embodiments, wherein said polypeptide has no killing activity against Lactobacilli crispatus, Lactobacilli gasseri, and/or Lactobacilli jensenii.
9. The pharmaceutical composition according to any one of the preceding embodiments, wherein said excipient is present at a concentration of about 0.01% and 10%, preferably about 0.1% to 1%, more preferably about 0.2% to 0.5%, most preferably about 0.25% or about 0.35%.
10. The pharmaceutical composition according to any one of the preceding embodiments, wherein the polyacrylic acid polymer is a crosslinked polyacrylic acid polymer or a carbomer.
11. The pharmaceutical composition according to any one of the preceding embodiments, wherein the combination of said active agent with said excipient increases the pharmaceutical activity of said active agent.
12. The pharmaceutical composition according to any one of the preceding embodiments, wherein said composition is suitable for intra-vaginal delivery of the active agent.
13. The pharmaceutical composition according to any one of the preceding embodiments, for use in treating a bacterial infection in a subject, preferably wherein the bacterial infection is bacterial vaginosis.
14. The pharmaceutical composition for use according to embodiment 13, wherein the subject previously failed a treatment with antibiotics and/or wherein the infective bacteria are resistant to a treatment with antibiotics.
15. The pharmaceutical composition for use according to embodiment 14, wherein said antibiotics are nitroimidazoles and/or Clindamycin.
16. The pharmaceutical composition for use according to embodiment 14 or 15, wherein the antibiotics are Metronidazole, Tinidazole, Secnidazole, Clindamycin or any combination thereof.
17. The pharmaceutical composition for use according to any one of embodiments 13 to 16, wherein said subject suffers from recurrent bacterial vaginosis, preferably wherein said subject had two or more episodes of BV in 6 months or had three or more episodes of BV in 12 months.
18. The pharmaceutical composition for use according to any one of embodiments 13 to 17, which is to be administered locally into the vagina of a female subject and/or into or on the glans penis, prepuce or urethral entry of a male subject.
19. Use of the pharmaceutical composition according to any one of embodiments 1 to 12 for the manufacture of a medicament for the treatment or the prevention of a bacterial infection, preferably bacterial vaginosis.
20. A method of treating or preventing a bacterial infection, preferably bacterial vaginosis, in a subject in need thereof comprising administering to said subject a therapeutically effective amount of the pharmaceutical composition according to any one of embodiments 1 to 12.

That is, the present invention is based on the surprising and unexpected finding that polyacrylic acid polymers can increase the killing activity of bactericidal polypeptides against bacteria of the genus Gardnerella. In the appended Example 2, it was shown that formulations of the bactericidal polypeptides H2B10 (PM-477) and H2B10B11 with a polyacrylic acid polymer resulted in killing activity against Gardnerella biofilms that was greater than the added killing activities of the individual components alone, clearly indicating a synergistic mode of action.

While polyacrylic acid polymers alone show some degree of killing activity against Gardnerella biofilms (see Example 2), polyacrylic acid polymers alone have no killing activity against planktonic Gardnerella cells (Example 4, FIG. 4) and only very limited killing activity against other BV-associated pathogens (Example 4, FIG. 5). Despite this limited or absent killing activity of polyacrylic acid polymers against planktonic bacteria, it was surprisingly demonstrated in appended Examples 3 and 5 (FIGS. 3A-3C and 6) that polyacrylic acid polymers improve the killing activity of bactericidal polypeptides against planktonic Gardnerella species, and also against other pathogens associated with bacterial vaginosis, such as Lactobacillus iners, Mobiluncus mulieris, Atopobium vaginae, and Prevotella bivia. Of note, the bactericidal polypeptide alone has no killing activity against other pathogens associated with bacterial vaginosis (FIG. 1B). It was thus surprisingly found that polyacrylic acid polymers cannot only improve the killing activity of bactericidal polypeptides against Gardnerella species, but also increased the antimicrobial spectrum of bactericidal polypeptides to target other pathogens associated with bacterial vaginosis. It is worth noting that this surprising effect was observed for two different polyacrylic acid polymers, i.e., a linear polyacrylic acid polymer and a crosslinked polyacrylic acid polymer (Carbopol 974P).

Thus, compelling evidence has been provided that formulating bactericidal polypeptides, in particular Gardnerella-specific bactericidal polypeptides, with a polyacrylic acid polymer can significantly increase the killing activity of the bactericidal polypeptide against Gardnerella species and other BV-associated bacteria.

Accordingly, in a particular embodiment, the invention relates to a pharmaceutical composition comprising an effective amount of an active agent and a pharmaceutical acceptable excipient, wherein said active agent is a polypeptide having a killing activity against Gardnerella and wherein said excipient is a polyacrylic acid polymer.

The active agent comprised in the pharmaceutical composition of the present invention is preferably a bactericidal polypeptide which is Gardnerella-genus specific, i.e., it specifically targets bacteria that belong to the genus Gardnerella. The polypeptide comprised in the pharmaceutical composition of the present invention thus preferably has killing activity against species in the genus Gardnerella. For example, the polypeptide comprised in the pharmaceutical composition of the present invention may have killing activity against Gardnerella vaginalis sensu stricto, Gardnerella leopoldii, Gardnerella piotii and/or Gardnerella swidsinskii, preferably against all of them. The killing activity of the polypeptide comprised in the pharmaceutical composition of the present invention against Gardnerella is more preferably a genus-selective killing activity against Gardnerella.

Herein “genus-selective killing activity” or “genus-specific bacteriolytic effect” means that the polypeptide comprised in the pharmaceutical composition of the present invention does not have killing activity or bacteriolytic effect against bacteria in general. Preferably, the polypeptide comprised in the pharmaceutical composition of the present invention has genus-selective killing activity against Gardnerella, but not against Lactobacilli crispatus, Lactobacilli gasseri, and/or Lactobacilli jensenii. More preferably, said polypeptide has no killing activity against all of these Lactobacilli, i.e., Lactobacilli crispatus, Lactobacilli gasseri, and Lactobacilli jensenii.

The skilled person will understand that the term “genus-selective killing activity” is not to be understood in the strictest sense in view of the overwhelming diversity of bacteria and the uncertain boundaries between different species and genera. For example, the species Lactobacillus iners is classified as a species of the genus Lactobacillus, although it significantly differs from other species of the genus Lactobacillus, such as Lactobacillus crispatus, which encodes more than twice as many proteins than Lactobacillus iners (see France et al., Applied and Environmental Microbiology. 82 (24): 7063-7073). It is therefore to be understood that an endolysin with “genus-selective killing activity” can have lytic activity against bacterial species of other genera, but preferably has no killing activity against the majority of bacteria.

In certain embodiments, the polypeptide comprised in the pharmaceutical composition of the present invention may have killing activity against other pathogens that are associated with BV. That is, in certain embodiments, the polypeptide comprised in the pharmaceutical composition of the present invention may have killing activity against one or more of Lactobacillus iners, Mobiluncus mulieris, Atopobium vaginae, and/or Prevotella bivia, in particular when formulated with a polyacrylic acid polymer. In certain embodiments, the polypeptide comprised in the pharmaceutical composition of the present invention may have killing activity against all of Lactobacillus iners, Mobiluncus mulieris, Atopobium vaginae, and Prevotella bivia, in particular when formulated with a polyacrylic acid polymer.

As used herein, the “killing activity” of an active agent, such as a bactericidal polypeptide, against particular bacteria can be defined as a reduction in the number of viable bacteria cells caused by the activity of said active agent. The killing activity of the active agent against said bacteria can be complete meaning that 100% of the bacterial cells have been killed or partial meaning that at least about 80%, at least about 90%, at least about 95%, at least about 99%, or at least about 99.9% of the bacterial cells have been killed. The killing activity of an active agent on a particular microorganism may be determined by standard procedures in the field including those based on the determination of the Minimum Inhibitory Concentrations (MICs) of an antimicrobial agent defined as the lowest concentration of said antimicrobial agent that inhibits the visible growth of a microorganism after overnight incubation as described in Andrews, 2001, J Antimicrobial Chemotherapy, 48, Suppl. SI, 5-16 or in “Document M7-A7, Methods for dilution antimicrobial susceptibility tests for bacteria that grow aerobically; Approved standards, 7th Edition, January 2006, vol. 26, No. 2” published by Clinical and Laboratory Standards Institute. Another suitable method for determining the killing activity of an active agent is described in the example section of WO 2020/225335 and consists of measuring a decrease in optical density at 610-620 nm of a bacterial cell suspension and/or a decrease in Colony Forming Units (CFU) per milliliter of a bacterial cell suspension after exposure to the active agent to be tested. The decrease of the Optical Density measured at 610-620 nm of a suspension of bacteria, the susceptibility of which is to be tested, can be determined in an in vitro turbidity assay performed in presence of the active agent. According to another embodiment, in an in vitro turbidity test, an active agent has killing activity against Gardnerella when said active agent decreases the OD (610-620 nm) of a suspension of at least one strain of Gardnerella bacteria by more than 20%, more than 30%, more than 40%, more than 50%, more than 60%, more than 70%, more than 80%, more than 90%, or more than 95%.

The polypeptide comprised in the pharmaceutical composition of the present invention further preferably has killing activity against antibiotic resistant Gardnerella strains, as described in more detail herein below. In one preferred embodiment, the polypeptide comprised in the pharmaceutical composition of the present invention has killing activity against Gardnerella strains which are resistant to one or more antibiotics selected from the group consisting of the nitroimidazoles and Clindamycin. In one more preferred embodiment, the polypeptide comprised in the pharmaceutical composition of the present invention has killing activity against Gardnerella strains which are resistant to Metronidazole, Tinidazole, Secnidazole, Clindamycin or any combination thereof. In one even more preferred embodiment, the polypeptide comprised in the pharmaceutical composition of the present invention has killing activity against Gardnerella strains which are resistant to Metronidazole, and/or, Clindamycin. In one even more preferred embodiment, the polypeptide comprised in the pharmaceutical composition of the present invention has killing activity against Gardnerella strains which are resistant to Metronidazole. In one more preferred embodiment, the polypeptide comprised in the pharmaceutical composition of the present invention has killing activity against Gardnerella strains which are highly resistant to said antibiotics mentioned above.

The bactericidal polypeptide comprised in the pharmaceutical composition according to the invention may be any polypeptide that has killing activity against one or more Gardnerella species. Thus, in a particular embodiment, the invention relates to the pharmaceutical composition according to the invention, wherein said polypeptide has a killing activity against Gardnerella vaginalis sensu stricto, Gardnerella leopoldii, Gardnerella piotii and/or Gardnerella swidsinskii, or any other species in the genus Gardnerella. Means and method for analyzing whether a given polypeptide has killing activity against Gardnerella species are provided herein and are known to the person skilled in the art.

The pharmaceutical composition according to the invention is preferably used in the treatment of bacterial vaginosis. In certain embodiments, the pharmaceutical composition may be effective in killing other bacteria that are associated with bacterial vaginosis, such as Lactobacillus iners, Atopobium vaginae, Prevotella bivia, Mubiluncus mulieris, Mubiluncus curtisii, and Streptococcus agalactiae, in particular when the pharmaceutical composition comprises a polyacrylic acid polymer. Thus, in a particular embodiment, the invention relates to the pharmaceutical composition according to the invention, wherein said polypeptide has killing activity against Lactobacillus iners, Atopobium vaginae, Prevotella bivia, Mubiluncus mulieris, Mubiluncus curtisii, and/or Streptococcus agalactiae, in particular when formulated with a polyacrylic acid polymer.

While it is preferred herein that the polypeptide comprised in the pharmaceutical composition of the invention has killing activity against Gardnerella species and other pathogens associated with BV, it is important that the polypeptide comprised in the pharmaceutical composition of the invention has no killing activity, or essentially no killing activity, against commensal vaginal bacteria. Commensal vaginal bacteria are important for maintaining a normal vaginal flora and include, without limitation, Lactobacilli crispatus, Lactobacilli gasseri, and Lactobacilli jensenii. It has been demonstrated in appended Example 3 that the bactericidal polypeptide H2B10 (PM-477) and in appended Example 5 that the bactericidal polypeptide H2B10B11 has no killing activity against the commensal vaginal bacterium Lactobacilli crispatus, which is one of the most prevalent bacteria in a healthy vaginal microbiota. Thus, in a particular embodiment, the invention relates to the pharmaceutical composition according to the invention, wherein said polypeptide has no killing activity against Lactobacilli crispatus, Lactobacilli gasseri, and/or Lactobacilli jensenii. In certain embodiments, the invention relates to the pharmaceutical composition according to the invention, wherein said polypeptide has killing activity against Lactobacillus iners, but has no killing activity against Lactobacilli crispatus, Lactobacilli gasseri, and/or Lactobacilli jensenii.

That is, in certain embodiments, the polypeptide comprised in the pharmaceutical composition of the invention is an endolysin having selective killing activity against Gardnerella vaginalis sensu stricto, and at least one of Gardnerella leopoldii, Gardnerella piotii, Gardnerella swidsinskii, Lactobacillus iners, Atopobium vaginae, Prevotella bivia, Mubiluncus mulieris, Mubiluncus curtisii, and/or Streptococcus agalactiae; wherein the endolysin has no killing activity, or essentially no killing activity, against at least one of Lactobacilli crispatus, Lactobacilli gasseri, and/or Lactobacilli jensenii.

The terms “peptide”, “polypeptide”, “protein” and variations of these terms refer to peptides, oligopeptides, oligomers or proteins, including fusion proteins, comprising at least two amino acids joined to each other by a normal or modified peptide bond, such as in the cases of the isosteric peptides, for example. These terms also include herewith “peptidomimetics” which are defined as peptide analogs containing non-peptidic structural elements, which peptides are capable of mimicking or antagonizing the biological action(s) of a natural parent peptide. A peptidomimetic lacks classical peptide characteristics such as enzymatically scissile peptide bonds. A peptide or polypeptide can be composed of amino acids other than the 20 amino acids defined by the genetic code. It can be composed of L-amino acids and/or D-amino acids. A peptide or polypeptide can equally be composed of amino acids modified by natural processes, such as post-translational maturation processes or by chemical processes, which are well known to a person skilled in the art. Such modifications are fully detailed in the literature. These modifications can appear anywhere in the polypeptide: in the peptide skeleton, in the amino acid chain or even at the carboxy- or amino-terminal ends. A peptide or polypeptide can be branched following a ubiquitination or be cyclic with or without branching. This type of modification can be the result of natural or synthetic post-translational processes that are well known to a person skilled in the art. For example, peptide or polypeptide modifications can include acetylation, acylation, ADP-ribosylation, amidation, covalent fixation of a nucleotide or of a nucleotide derivative, covalent fixation of a lipid or of a lipidic derivative, the covalent fixation of a phosphatidylinositol, covalent or non-covalent cross-linking, cyclization, disulfide bond formation, demethylation, glycosylation including pegylation, hydroxylation, iodization, methylation, myristoylation, oxidation, proteolytic processes, phosphorylation, prenylation, racemization, seneloylation, sulfatation, amino acid addition such as arginylation or ubiquitination. Such modifications are fully detailed in the literature and well-known by the skilled person of the art.

In preferred embodiments of the present invention, the polypeptide comprised in the pharmaceutical composition according to the invention is an endolysin. Thus, in a particular embodiment, the invention relates to the pharmaceutical composition according to the invention, wherein said polypeptide is an endolysin having a killing activity against Gardnerella.

The term “endolysin” as used herein refers to a polypeptide usually produced by bacteriophages to digest the host bacteria cell wall and release bacteriophage progeny. An endolysin is a cell-wall lytic enzyme encoded by bacteriophages which has the ability to hydrolyze the cell-wall of target bacteria when added exogenously (lysis-from-without). This novel class of antibacterials has important advantages over classical antibiotics, e.g. a novel mode of action; a narrow spectrum of susceptible bacteria; rapid killing of both stationary- and exponentially-growing bacteria; activity on mucous membranes and bacterial biofilms; low probability of developing resistances; and reduced impact on normal microbiota. These unique features have boosted the interest on the biotechnological and pharmacological exploitation of lysins and their recent inclusion among the top current alternatives to fight antibiotic resistances.

Endolysins often consist of two or more domains: at least one catalytic domain, such as a hydrolase domain (typically located at the N-terminal of the polypeptide), which cleaves specific motifs in the peptidoglycan layer, and often one or more cell wall binding domains (classically located at the C-terminal of the polypeptide), which is involved in the specific binding and processing of the bacterial peptidoglycan. Although providing a general organization for endolysin structure, this typical architecture is not a defined characteristic of all endolysins. Endolysins from Gram-positive bacteria and their phages usually comprise at least one catalytic domain and one or more cell wall-binding domains. In contrast, many endolysins produced by Gram-negative species or their phages only contain the catalytic domain, though modular endolysins have also been reported. The catalytic units dictate the type of peptidoglycan (PG) bond to be cleaved, whereas the cell wall-binding domain(s) largely determines the lytic spectrum by specific recognition of cell wall elements distributed in genus-, or species/strain-specific manner.

The endolysin comprised in the pharmaceutical composition of the present invention is preferably a functional polypeptide, wherein the function comprises specifically targeting bacteria from the Gardnerella genus, more preferably specially killing bacteria from the Gardnerella genus. The endolysin comprised in the pharmaceutical composition of the present invention further preferably comprises a catalytic domain or a functional fragment thereof and/or a cell wall binding domain or a functional fragment thereof. The endolysin comprised in the pharmaceutical composition of the present invention might be a natural or a recombinant endolysin. The endolysin comprised in the pharmaceutical composition of the present invention most preferably a recombinant endolysin. The endolysin comprised in the pharmaceutical composition of the present invention is more preferably a recombinant endolysin comprising or consisting of
(i) a N-terminal catalytic domain, or a functional variant thereof;
(ii) a C-terminal cell-wall binding region, or a functional variant thereof, wherein the C-terminal cell-wall binding region comprises or consists of at least one cell-wall binding domain; and
(iii) optionally a linker region between the N-terminal catalytic domain and the C-terminal cell-wall binding region,
   and has preferably a killing activity, more preferably genus-selective killing activity, against Gardnerella cells/strains.

As defined herewith the terms “binding” and “bind to” referring to the binding capacity of an endolysin to the cell wall of a particular bacteria refers to the ability of said endolysin to specifically interact and adhere to the cell wall of said bacteria. The binding capacity of an endolysin to the cell wall of a bacteria can be determined by methods know of the art.

In the context of the present disclosure, the term “recombinant endolysin” preferably refers to an endolysin which has been domain-swapped, as defined in WO 2020/225335. In line with this definition, the person skilled in the art readily understands that the “domain-swapped” or “recombinant” endolysins as described herein are non-naturally occurring endolysins. That is, the recombinant endolysin for use of the present invention has been modified by hand of man and excludes, by definition, natural endolysins, i.e., as it can be naturally found in nature. The appended examples as well as the teaching of WO 2020/225335 provide suitable method(s) how to generate the artificial endolysin of the invention.

The term “catalytic domain” or “enzymatic domain” refer to the part of the protein chain which contains the region where the catalyzed chemical reaction takes place. The “catalytic domain” as used herein refers to a functional polypeptide, wherein the function comprises the ability to lyse the cell wall of Gardnerella. In particular, the catalytic domain as described herein can preferably modify and/or cleave a substrate in Gardnerella cell walls, preferably peptidoglycan. Preferably, the catalytic domain cleaves peptidoglycan in Gardnerella cell walls and can cause Gardnerella cell lysis. Preferably, the catalytic domain can modify and/or cleave bonds that are present in the cell wall and/or peptidoglycan of Gardnerella spp., such as Gardnerella vaginalis sensu stricto, Gardnerella leopoldii, Gardnerella piotii and/or Gardnerella swidsinskii, preferably all of them. Suitably, the catalytic domain does not modify and/or cleave a substrate, preferably peptidoglycan, present in cell wall of bacteria other than Gardnerella spp., preferably healthy vaginal commensal bacteria, such as Lactobacillus spp. including Lactobacilli crispatus, Lactobacilli gasseri, and/or Lactobacilli jensenii. The catalytic domain may be a N-acetylmuramidase, N-acetylmuramoyl-L-alanine amidases, L-alanoyl-D-glutamate endopeptidases, interpeptide bridge endopeptidases or N-acetyl-beta-D-glucosaminidases. Preferably, the N-terminal catalytic domain is a N-acetylmuramidase, most preferably a 1,4-beta-N-acetylmuramidase. The catalytic domain is preferably located N-terminally within the (recombinant) endolysin, thereby referred to as “N-terminal catalytic domain”, even more preferably the N-terminal catalytic domain is located N-terminally from the C-terminal cell-wall binding region within the (recombinant) endolysin.

The endolysin comprised in the pharmaceutical composition of the present invention preferably comprises a catalytic domain consisting of a polypeptide comprising or consisting of the amino acid sequence of any one of SEQ ID NOs: 2 to 10 or any functional variant thereof having at least 80% identity with the amino acid sequence of any one of SEQ ID NOs: 2 to 10. As shown in WO 2020/225335, the most active catalytic domain is “H2” (SEQ ID NO: 3). Accordingly, in a preferred aspect of the present invention the catalytic domain is consisting of a polypeptide which comprises or consists of the amino acid sequence of SEQ ID NO: 3, or any functional variant thereof having at least 80% identity (preferably at least 85% identity, more preferably at least 90% identity, even more preferably at least 95% identity, even more preferably at least 96% identity, even more preferably at least 97% identity, even more preferably at least 98% identity, even more preferably at least 99% identity, even more preferably at least 99.5% identity, and most preferably at least 99.7% identity) with the amino acid sequence of SEQ ID NO: 3, whereby the endolysin is functional, wherein the function comprises the ability to lyse the cell wall of Gardnerella. The catalytic domain is preferably located N-terminally within the (recombinant) endolysin, thereby referred to as “N-terminal catalytic domain”, even more preferably the (recombinant) endolysin further comprises a cell-wall binding region and the N-terminal catalytic domain is located N-terminally from the C-terminal cell-wall binding region within the (recombinant) endolysin.

The “cell-wall binding region” as used herein refers to a functional polypeptide, wherein the function comprises the ability to bind to the cell wall of Gardnerella. The cell-wall binding region may comprise or consist of one, two, three or more cell-wall binding domains. Cell wall binding domains are polypeptides that interact with and/or bind to bacteria cell walls and/or specific substrates within bacteria cell walls. In particular, the cell wall binding domain described herein can preferably specifically bind to the cell wall (e.g., to the peptidoglycan) of Gardnerella spp., such as Gardnerella vaginalis sensu stricto, Gardnerella leopoldii, Gardnerella piotii and/or Gardnerella swidsinskii, preferably all of them. The cell-wall binding region is preferably located C-terminally within the (recombinant) endolysin, thereby referred to as “C-terminal cell-wall binding region”, even more preferably the C-terminal cell-wall binding region is located C-terminally from the N-terminal catalytic domain within the (recombinant) endolysin.

The endolysin comprised in the pharmaceutical composition of the present invention preferably comprises a cell-wall binding region comprising or consisting of at least one cell-wall binding domain selected from the group consisting of polypeptides comprising or consisting of the amino acid sequence of any one of SEQ ID NOs: 11 to 28, and any functional variant thereof having at least 80% identity (preferably at least 85% identity, more preferably at least 90% identity, even more preferably at least 95% identity, even more preferably at least 96% identity, even more preferably at least 97% identity, even more preferably at least 98% identity, even more preferably at least 99% identity, even more preferably at least 99.5% identity, and most preferably at least 99.7% identity) with the amino acid sequence of any one of SEQ ID NOs: 11 to 28. As shown in WO 2020/225335, the most active cell-wall binding region is “B10” (comprising the cell-wall binding domains of SEQ ID NOs: 23 and 24), followed by “B11” (comprising the cell-wall binding domains of SEQ ID NOs: 25 and 26). Thus, in a preferred aspect of the present invention the cell-wall binding domain(s) of is/are selected from the group consisting of polypeptides comprising or consisting of the amino acid sequence of any one of SEQ ID NOs: 23, 24, 25 and 26, and any functional variant thereof having at least 80% identity (preferably at least 85% identity, more preferably at least 90% identity, even more preferably at least 95% identity, even more preferably at least 96% identity, even more preferably at least 97% identity, even more preferably at least 98% identity, even more preferably at least 99% identity, even more preferably at least 99.5% identity, and most preferably at least 99.7% identity) with the amino acid sequence of any one of SEQ ID NOs: 23, 24, 25 and 26, whereby the endolysin is functional, wherein the function comprises the ability to lyse the cell wall of Gardnerella. The cell-wall binding region is preferably located C-terminally within the (recombinant) endolysin, thereby referred to as “C-terminal cell-wall binding region”, even more preferably the (recombinant) endolysin further comprises a catalytic domain and the catalytic domain is located N-terminally from the C-terminal cell-wall binding region within the (recombinant) endolysin.

The endolysin comprised in the pharmaceutical composition of the present invention comprises preferably two cell-wall binding domains (within the cell-wall binding region). In one preferred aspect of the present invention the cell-wall binding domains of the endolysin of the invention each consists of a polypeptide comprising or consisting of the amino acid sequence of any one of SEQ ID NOs: 23, 24, 25 and 26, and any functional variant thereof having at least 80% identity (preferably at least 85% identity, more preferably at least 90% identity, even more preferably at least 95% identity, even more preferably at least 96% identity, even more preferably at least 97% identity, even more preferably at least 98% identity, even more preferably at least 99% identity, even more preferably at least 99.5% identity, and most preferably at least 99.7% identity) with the amino acid sequence of any one of SEQ ID NOs: 23, 24, 25 and 26, whereby the endolysin is functional, wherein the function comprises the ability to lyse the cell wall of Gardnerella. In one even more preferred aspect of the present invention, the endolysin comprises a first cell-wall binding domain and a second cell-wall binding domain, wherein said first cell-wall binding domain is selected from the group consisting of SEQ ID NOs: 23 and 25, and said second cell-wall binding domain is selected from the group consisting of SEQ ID NOs: 24 and 26. Preferably, said first cell-wall binding domain is located N-terminally of said second cell-wall binding domain.

In one more preferred embodiment, the endolysin comprised in the pharmaceutical composition of the present invention comprises
(i) a N-terminal catalytic domain consisting of a polypeptide which comprises or consists of the amino acid sequence of SEQ ID NO: 3, or any functional variant thereof having at least 80% identity (preferably at least 85% identity, more preferably at least 90% identity, even more preferably at least 95% identity, even more preferably at least 96% identity, even more preferably at least 97% identity, even more preferably at least 98% identity, even more preferably at least 99% identity, even more preferably at least 99.5% identity, and most preferably at least 99.7% identity) with the amino acid sequence of SEQ ID NO: 3; and
(ii) a C-terminal cell-wall binding region comprising or consisting of a first cell-wall binding domain and a second cell-wall binding domain,
   wherein said first cell-wall binding domain is selected from the group consisting of SEQ ID NOs: 23 and 25, and any functional variant thereof having at least 80% identity (preferably at least 85% identity, more preferably at least 90% identity, even more preferably at least 95% identity, even more preferably at least 96% identity, even more preferably at least 97% identity, even more preferably at least 98% identity, even more preferably at least 99% identity, even more preferably at least 99.5% identity, and most preferably at least 99.7% identity) with the amino acid sequence of any one of SEQ ID NOs: 23 and 25, and
   wherein said second cell-wall binding domain is selected from the group consisting of SEQ ID NOs: 24 and 26 and any functional variant thereof having at least 80% identity (preferably at least 85% identity, more preferably at least 90% identity, even more preferably at least 95% identity, even more preferably at least 96% identity, even more preferably at least 97% identity, even more preferably at least 98% identity, even more preferably at least 99% identity, even more preferably at least 99.5% identity, and most preferably at least 99.7% identity) with the amino acid sequence of any one of SEQ ID NOs: 24 and 26;
   whereby the endolysin is functional, wherein the function comprises the ability to lyse the cell wall of Gardnerella. Preferably, said first cell-wall binding domain is located N-terminally of said second cell-wall binding domain.

In one particularly preferred embodiment, the endolysin comprised in the pharmaceutical composition of the present invention comprises
(i) a N-terminal catalytic domain consisting of a polypeptide which comprises or consists of the amino acid sequence of SEQ ID NO: 3; and
(ii) a C-terminal cell-wall binding region comprising or consisting of a first cell-wall binding domain and a second cell-wall binding domain,
   wherein said first cell-wall binding domain is selected from the group consisting of SEQ ID NOs: 23 and 25, and said second cell-wall binding domain is selected from the group consisting of SEQ ID NOs: 24 and 26.

Preferably, said first cell-wall binding domain is located N-terminally of said second cell-wall binding domain.

Illustrative examples of particularly preferred endolysins comprised in the pharmaceutical composition of the present invention are “H2B10” (comprising from N-terminal to C-terminal: SEQ ID NOs: 3, 23, and 24), “H2B11” (comprising from N-terminal to C-terminal: SEQ ID NOs: 3, 25, and 26) as defined in WO 2020/225335, and “H2B10B11” (comprising from N-terminal to C-terminal: SEQ ID NOs: 3, 23, and 26).

The endolysin comprised in the pharmaceutical composition of the present invention further preferably comprises a linker region between the N-terminal catalytic domain and the C-terminal cell-wall binding region. The linker region may consist of a polypeptide having a length of 6 to 18 amino acids, preferably a length of 9 to 15 amino acids, even more preferably a length of 12 amino acids. Preferably, the linker region may consist of a polypeptide comprising or consisting of the amino acid sequence (i) (XXX)n, wherein each X can be independently G, A or S, preferably wherein the amino acid sequence (XXX)n is (GGS)n, wherein n corresponds to the number of repetitions of the sequence XXX, preferably wherein n is 2, 3, 4, 5 or 6, or (ii) X₁X₂GLNGX₃X₄NGGS (SEQ ID NO: 36), wherein X₁ is N or K, X₂ is A or V, X₃ is Y or C and X₄ is K or Q. Non-limiting examples of such linker regions are provided in SEQ ID NOs: 29 to 35.

In a particular preferred embodiment, the endolysin comprised in the pharmaceutical composition of the present invention is “H2B10”, the sequence of which is set forth in SEQ ID NO: 1 or is “H2B10B11” the sequence of which is set forth in SEQ ID NO: 37.

In a particular embodiment, the invention relates to the pharmaceutical composition according to the invention, wherein said polypeptide is a polypeptide having at least 80% sequence identity with the amino acid sequence as provided in SEQ ID NO: 1 and having a killing activity against Gardnerella.

That is, in certain embodiments, the invention provides a pharmaceutical composition comprising an effective amount of Gardnerella-specific endolysin, preferably a recombinant Gardnerella-specific endolysin, and a polyacrylic acid polymer, wherein said endolysin is a polypeptide having at least 80% sequence identity with the amino acid sequence as provided in SEQ ID NO: 1 and having a killing activity against Gardnerella. In one more preferred embodiment, the invention provides a pharmaceutical composition comprising an effective amount of Gardnerella-specific endolysin, preferably a recombinant Gardnerella-specific endolysin, and a polyacrylic acid polymer, wherein said endolysin is a polypeptide having at least 90% sequence identity with the amino acid sequence as provided in SEQ ID NO: 1 and having a killing activity against Gardnerella. In one even more preferred embodiment, the invention provides a pharmaceutical composition comprising an effective amount of Gardnerella-specific endolysin, preferably a recombinant Gardnerella-specific endolysin, and a polyacrylic acid polymer, wherein said endolysin is a polypeptide having at least 95% sequence identity with the amino acid sequence as provided in SEQ ID NO: 1 and having a killing activity against Gardnerella. In one even more preferred embodiment, the invention provides a pharmaceutical composition comprising an effective amount of Gardnerella-specific endolysin, preferably a recombinant Gardnerella-specific endolysin, and a polyacrylic acid polymer, wherein said endolysin is a polypeptide having at least 99% sequence identity with the amino acid sequence as provided in SEQ ID NO: 1 and having a killing activity against Gardnerella. In one most preferred embodiment, the invention provides a pharmaceutical composition comprising an effective amount of Gardnerella-specific endolysin, preferably a recombinant Gardnerella-specific endolysin, and a polyacrylic acid polymer, wherein said endolysin comprises or consists of the amino acid sequence as provided in SEQ ID NO: 1.

Accordingly, in a particular embodiment, the invention relates to the pharmaceutical composition according to the invention, wherein said polypeptide is the recombinant endolysin comprising the amino acid sequence provided in SEQ ID NO: 1.

The endolysin comprising or consisting of the amino acid sequence as provided in SEQ ID NO: 37 may also be employed in context of this invention, i.e., in context of a pharmaceutical composition that is suitable for treating a bacterial vaginosis comprising an effective amount of Gardnerella-specific endolysin, preferably a recombinant Gardnerella-specific endolysin, and a polyacrylic acid polymer. Accordingly, the endolysin comprising or consisting of the amino acid sequence as provided in SEQ ID NO: 37 is an alternative to the Gardnerella-specific endolysin provided in SEQ ID NO. 1.

Thus, in a particular embodiment, the invention relates to the pharmaceutical composition according to the invention, wherein said polypeptide is a polypeptide having at least 80% sequence identity with the amino acid sequence as provided in SEQ ID NO: 37 and having a killing activity against Gardnerella.

Thus, in certain embodiments, the invention provides a pharmaceutical composition comprising an effective amount of Gardnerella-specific endolysin, preferably a recombinant Gardnerella-specific endolysin, and a polyacrylic acid polymer, wherein said endolysin is a polypeptide having at least 80% sequence identity with the amino acid sequence as provided in SEQ ID NO: 37 and having a killing activity against Gardnerella. In one more preferred embodiment, the invention provides a pharmaceutical composition comprising an effective amount of Gardnerella-specific endolysin, preferably a recombinant Gardnerella-specific endolysin, and a polyacrylic acid polymer, wherein said endolysin is a polypeptide having at least 90% sequence identity with the amino acid sequence as provided in SEQ ID NO: 37 and having a killing activity against Gardnerella. In one even more preferred embodiment, the invention provides a pharmaceutical composition comprising an effective amount of Gardnerella-specific endolysin, preferably a recombinant Gardnerella-specific endolysin, and a polyacrylic acid polymer, wherein said endolysin is a polypeptide having at least 95% sequence identity with the amino acid sequence as provided in SEQ ID NO: 37 and having a killing activity against Gardnerella. In one even more preferred embodiment, the invention provides a pharmaceutical composition comprising an effective amount of Gardnerella-specific endolysin, preferably a recombinant Gardnerella-specific endolysin, and a polyacrylic acid polymer, wherein said endolysin is a polypeptide having at least 99% sequence identity with the amino acid sequence as provided in SEQ ID NO: 37 and having a killing activity against Gardnerella. In one most preferred embodiment, the invention provides a pharmaceutical composition comprising an effective amount of Gardnerella-specific endolysin, preferably a recombinant Gardnerella-specific endolysin, and a polyacrylic acid polymer, wherein said endolysin comprises or consists of the amino acid sequence as provided in SEQ ID NO: 37.

Accordingly, in a particular embodiment, the invention relates to the pharmaceutical composition according to the invention, wherein said polypeptide is the recombinant endolysin comprising the amino acid sequence provided in SEQ ID NO: 37.

In one aspect, the invention relates to a pharmaceutical composition comprising a recombinant endolysin having at least 80% sequence identity with the amino acid sequence as provided in SEQ ID NO: 1 or 37 and having killing activity against Lactobacillus iners for use in the treatment of a bacterial infection involving and/or caused by the species Lactobacillus iners.

L. iners is a bacterial species associated with an imbalanced vaginome, and is known as being potentially relevant in case of BV. After metronidazole (MDZ) treatment, patients often transition into a L. iners-dominated microbiome, followed by recurrence of BV. Therefore, endolysins having (additional) killing activity against L. iners are of particular relevance in the treatment of BV.

Preferably, the invention relates to a pharmaceutical composition comprising a recombinant endolysin comprising an amino acid sequence as provided in SEQ ID NO: 1 or 37 for use in the treatment of a bacterial infection involving and/or caused by the species Lactobacillus iners. It has been surprisingly shown in FIGS. 1B, 3A-C and 6 that the endolysins PM-477 and H2B10B11 have killing activity against L. iners, both in the absence and presence of a polyacrylic acid polymer. In certain embodiments, the bacterial infection caused by L. iners is a vaginal infection, in particular bacterial vaginosis. It is to be understood that any of the embodiments disclosed herein apply equally to the composition for use in the treatment of L. iners infections. In particular, the composition may further comprise a polyacrylic acid polymer as disclosed elsewhere herein. In certain embodiments, the invention relates to a method for treating infections involving and/or caused by L. iners in a subject in need, the method comprising a step of administering a recombinant endolysin having at least 80% sequence identity with the amino acid sequence as provided in SEQ ID NO: 1 or 37 and having killing activity against L. iners to said patient. In certain embodiments, the invention relates to a method for reducing the number of L. iners in in the vaginome of a subject in need, the method comprising a step of administering a recombinant endolysin having at least 80% sequence identity with the amino acid sequence as provided in SEQ ID NO: 1 or 37 and having killing activity against L. iners to said patient.

The term “variant” refers to a polypeptide including insertions, deletions, and/or substitutions, either non-conservative or preferably conservative, relative to the native amino acid sequence. For example, the polypeptide may comprise an amino acid sequence with at least 80% identity to the native amino acid sequence, preferably at least 85% identity, more preferably at least 90% identity, even more preferably at least 95% identity, even more preferably at least 96% identity, even more preferably at least 97% identity, even more preferably at least 98% identity, even more preferably at least 99% identity, even more preferably at least 99.5% identity, and most preferably at least 99.7% identity to said amino acid sequence. Percent identity can be determined by methods well known in the art, using suitable computer programs for example MatGAT 2.0 (Myers and Miller, CABIOS (1989). Preferably, % identity is identified over the whole lengths of the sequences to be compared. It will be appreciated that percent identity is calculated in relation to polypeptides whose sequence has been aligned optimally. Fragment and variants of an amino acid sequence may be made using any of the methods of protein engineering, directed evolution and/or site-directed mutagenesis well known in the art (for example, see Molecular Cloning: a Laboratory Manual, 3rd edition, Sambrook & Russell, 2001, Cold Spring Harbor Laboratory Press). It will be appreciated by skilled persons that a polypeptide according to the invention, or fragment, variant, or fusion thereof, may comprise or consist of a derivative of a native amino acid sequence, or a fragment or variant thereof. Chemical derivatives of one or more amino acids may be achieved by reaction with a functional side group. Such derivatised molecules include, for example, those molecules in which free amino acid groups have been derivatised to form amine hydrochlorides, p-toluene sulphonyl groups, carboxybenzoxy groups, f-butyloxycarbonyl groups, chloroacetyl groups or formyl groups. Free carboxyl groups may be derivatised to form salts, methyl and ethyl esters or other types of esters and hydrazides. Free hydroxyl groups may be derivatised to form O-acyl or O-alkyl derivatives. Also included as chemical derivatives are those peptides which contain naturally occurring amino acid derivatives of the twenty standard amino acids. For example: 4-hydroxyproline may be substituted for proline; 5-hydroxylysine may be substituted for lysine; 3-methylhistidine may be substituted for histidine; homoserine may be substituted for serine and ornithine for lysine. Derivatives also include peptides containing one or more additions or deletions as long as the requisite activity is maintained. Other included modifications are amidation, amino terminal acylation (e.g., acetylation or thioglycolic acid amidation), terminal carboxylamidation (e.g., with ammonia or methylamine), and the like terminal modifications. It will be further appreciated by persons skilled in the art that peptidomimetic compounds may also be useful. Thus, by ‘polypeptide’ we include peptidomimetic compounds which exhibit endolysin activity. The term ‘peptidomimetic’ refers to a compound that mimics the conformation and desirable features of a particular polypeptide as a therapeutic agent.

The composition according to the invention can contain one or more endolysin polypeptides. In certain embodiments, endolysin polypeptides can either be present as independent polypeptides or as fusion proteins comprising said endolysin polypeptides or fragments thereof.

The term “pharmaceutical composition” refers to a preparation which is in such a form as to permit biological activity of the active ingredient(s) to be unequivocally effective and which contains no additional component which would be toxic to patients to which the said composition would be administered. As used herein, “pharmaceutical composition” means a therapeutically effective formulation for use in the methods of the invention. A “therapeutically effective amount”, or “effective amount”, or “therapeutically effective”, as used herein, refers to that amount which provides a therapeutic effect for a given condition and administration regimen. This is a predetermined quantity of active material calculated to produce a desired therapeutic effect in association with the required additive and diluent, i.e., a carrier or administration vehicle. Further, it is intended to mean an amount sufficient to reduce, and most preferably prevent, a clinically significant deficit in the activity, function and response of the host. Alternatively, a therapeutically effective amount is sufficient to cause an improvement in a clinically significant condition in a host. As is appreciated by those skilled in the art, the amount of a compound may vary depending on its specific activity. Suitable dosage amounts may contain a predetermined quantity of active composition calculated to produce the desired therapeutic effect in association with the required diluent. In the methods and use for manufacture of compositions of the invention, a therapeutically effective amount of the active component is provided. A therapeutically effective amount can be determined by the ordinary skilled medical or veterinary worker based on patient characteristics, such as age, weight, sex, condition, complications, other diseases, etc., as is well known in the art.

In one embodiment of the invention, the pharmaceutical composition comprises a Gardnerella-specific endolysin as described herein and is for use in treating bacterial vaginosis as described herein. Thus, the pharmaceutical composition may comprise an amount of an endolysin, or fragment, variant, fusion or derivative thereof, sufficient to inhibit at least in part the growth of cells of the genus Gardnerella in a patient as described herein above who is infected or susceptible to infection with such cells. Preferably, the pharmaceutical composition comprises an amount of endolysin, or fragment, variant, fusion or derivative thereof, sufficient to kill cells of the genus Gardnerella in the patient to be treated as defined herein above.

The pharmaceutical composition according to the invention further comprises a pharmaceutically acceptable excipient. The term “pharmaceutically acceptable excipient” refers to an excipient comprised of a material that is not biologically or otherwise undesirable. The term “carrier” refers to any components present in a pharmaceutical formulation other than the active agent and thus includes excipients, diluents, binders, lubricants, disintegrants, fillers, coloring agents, wetting or emulsifying agents, pH buffering agents, preservatives and the like.

It was surprisingly found by the inventors that certain excipients, namely polyacrylic acid polymers can increase the killing activity of bactericidal polypeptides, in particular Gardnerella-specific endolysins. Thus, in a particular embodiment, the invention relates to the pharmaceutical composition according to the invention, wherein the combination of said active agent with said excipient increases the pharmaceutical activity of said active agent.

Within the present invention, an excipient, in particular a polyacrylic acid polymer, “increases the pharmaceutical activity” of the active agent, i.e., the Gardnerella-specific bactericidal polypeptide, if it increases the killing activity of said active agent against bacteria of the species Gardnerella. The excipient may increase the pharmaceutical activity of the active agent by at least 10%, at least 20%, at least 30%, at least 40%, at least 50%, at least 60%, at least 70%, or at least 80%. It can be seen in FIG. 2 that the excipient PAA increases the killing activity of the active agent PM-477 against Gardnerella biofilms by almost 100% and the killing activity of the active ingredient H2B10B11 against Gardnerella biofilms to an even greater extent.

The term “polyacrylic acid polymer” as used herein describes polymers of acrylic acid and includes homopolymers, copolymers and crosslinked polymers of acrylic acid. That is, a polyacrylic acid polymer is a polymer comprising or consisting of the formula (CH₂—CHCO₂H)ₙ.

In certain embodiments, the polyacrylic acid polymer is a linear polyacrylic acid polymer, in particular a linear polyacrylic acid homopolymer. It has been demonstrated in Example 2 that such a polymer significantly increases the killing activity of endolysins against Gardnerella biofilms. Furthermore, it was demonstrated in Example 3 that a linear polyacrylic acid homopolymer can extend the antimicrobial spectrum of the endolysin PM-477 to other BV-associated bacteria.

In certain embodiments, the “polyacrylic acid polymer” is a crosslinked polyacrylic acid polymer, such as a carbomer or a carboxyvinyl polymer. A crosslinked polyacrylic acid polymer is a homopolymer or copolymer of acrylic acid that is crosslinked with at least one other polymer. The term “carbomer” refers to a series of polymers of acrylic acid that are commonly used in pharmaceutical compositions. Carbomers are high molecular weight homopolymers or copolymers of acrylic acid that are crosslinked with polyalkenyl ethers of sugars or polyalcohols. The carbomer comprised in the pharmaceutical composition according to the present invention is preferably a carbomer that follows the US and/or European Pharmacopeia requirements. Carbomers are marketed under the trade name Carbopol. Thus, in a particular embodiment, the invention relates to the pharmaceutical composition according to the invention, wherein said excipient is a carbomer, preferably a Carbopol.

In certain embodiments, the pharmaceutical composition according to the invention comprises a specific type of carbomer. In certain embodiments, the pharmaceutical composition according to the invention comprises a mixture of two or more different types of carbomers.

The polyacrylic acid polymer comprised in the pharmaceutical composition according to the invention may be defined based on one or more physicochemical properties.

Preferably, the polyacrylic acid polymer comprised in the pharmaceutical composition according to the invention is defined based on its viscosity. That is, the polyacrylic acid polymer comprised in the pharmaceutical composition according to the invention may have a viscosity ranging from 300-115,000 mPa's when in the form of a 0.5% (w/v) gel (pH 7.3-7.8 at 25° C.).

In certain embodiments, the polyacrylic acid polymer comprised in the pharmaceutical composition according is a type A carbomer according to US Pharmacopeia having a viscosity of 4,000-11,000 mPa's when in the form of a 0.5% (w/v) gel (pH 7.3-7.8 at 25° C.). In certain embodiments, the type A carbomer is Carbopol 981, Carbopol 971 or Carbopol71G.

In certain embodiments, the polyacrylic acid polymer comprised in the pharmaceutical composition according is a type B carbomer according to US Pharmacopeia having a viscosity of 25,000-45,000 mPa's when in the form of a 0.5% (w/v) gel (pH 7.3-7.8 at 25° C.). In certain embodiments, the type B carbomer is Carbopol 974P, Carbopol 984 or Carbopol 5984, preferably Carbopol 974P.

In certain embodiments, the polyacrylic acid polymer comprised in the pharmaceutical composition according is a type C carbomer according to US Pharmacopeia having a viscosity of 40,000-60,000 mPa's when in the form of a 0.5% (w/v) gel (pH 7.3-7.8 at 25° C.). In certain embodiments, the type C carbomer is Carbopol 980.

In certain embodiments, the polyacrylic acid polymer comprised in the pharmaceutical composition according to the invention has a viscosity of 300-115,000 mPa's when in the form of a 0.5% (w/v) gel (pH 7.3-7.8 at 25° C.). In certain embodiments, the polyacrylic acid polymer comprised in the pharmaceutical composition according to the invention has a viscosity of 300-60,000 mPa's when in the form of a 0.5% (w/v) gel (pH 7.3-7.8 at 25° C.). In certain embodiments, the polyacrylic acid polymer comprised in the pharmaceutical composition according to the invention has a viscosity of 300-45,000 mPa's when in the form of a 0.5% (w/v) gel (pH 7.3-7.8 at 25° C.). In certain embodiments, the polyacrylic acid polymer comprised in the pharmaceutical composition according to the invention has a viscosity of 300-11,000 mPa's when in the form of a 0.5% (w/v) gel (pH 7.3-7.8 at 25° C.). In certain embodiments, the polyacrylic acid polymer comprised in the pharmaceutical composition according to the invention has a viscosity of 300-4,000 mPa's when in the form of a 0.5% (w/v) gel (pH 7.3-7.8 at 25° C.). In certain embodiments, the polyacrylic acid polymer comprised in the pharmaceutical composition according to the invention has a viscosity of 300-2000 mPa's when in the form of a 0.5% (w/v) gel (pH 7.3-7.8 at 25° C.).

In certain embodiments, the polyacrylic acid polymer comprised in the pharmaceutical composition according to the invention is a linear polyacrylic acid homopolymer having a viscosity of 300-2000 mPa's when in the form of a 0.5% (w/v) gel (pH 7.3-7.8 at 25° C.).

In certain embodiments, the polyacrylic acid polymer comprised in the pharmaceutical composition according to the invention is a type B carbomer, such as Carbomer 974P, having a viscosity of 25000-45000 mPa's when in the form of a 0.5% (w/v) gel (pH 7.3-7.8 at 25° C.).

In a particular embodiment, the invention relates to the pharmaceutical composition according to the invention, wherein said excipient is present at a concentration of about 0.1% and 10%.

That is, the polyacrylic acid polymer may be present in the pharmaceutical composition of the invention at a concentration of about 0.1%, about 0.2%, about 0.25%, about 0.3%, about 0.35%, about 0.4%, about 0.5%, about 0.6%, about 0.7%, about 0.8%, about 0.9%, about 1%, about 1.1%, about 1.2%, about 1.3%, about 1.4%, about 1.5%, about 1.6%, about 1.7%, about 1.8%, about 1.9%, about 2%, about 2.1%, about 2.2%, about 2.3%, about 2.4%, about 2.5%, about 2.6%, about 2.7%, about 2.8%, about 2.9%, about 3%, about 3.1%, about 3.2%, about 3.3%, about 3.4%, about 3.5%, about 3.6%, about 3.7%, about 3.8%, about 3.9%, about 4%, about 4.1%, about 4.2%, about 4.3%, about 4.4%, about 4.5%, about 4.6%, about 4.7%, about 4.8%, about 4.9%, about 5%, about 5.1%, about 5.2%, about 5.3%, about 5.4%, about 5.5%, about 5.6%, about 5.7%, about 5.8%, about 5.9%, about 6%, about 6.1%, about 6.2%, about 6.3%, about 6.4%, about 6.5%, about 6.6%, about 6.7%, about 6.8%, about 6.9%, about 7%, about 7.1%, about 7.2%, about 7.3%, about 7.4%, about 7.5%, about 7.6%, about 7.7%, about 7.8%, about 7.9%, about 8%, about 8.1%, about 8.2%, about 8.3%, about 8.4%, about 8.5%, about 8.6%, about 8.7%, about 8.8%, about 8.9%, about 9%, about 9.1%, about 9.2%, about 9.3%, about 9.4%, about 9.5%, about 9.6%, about 9.7%, about 9.8%, about 9.9%, or about 10%.

In a particularly preferred embodiment, the invention relates to the pharmaceutical composition according to the invention, wherein said excipient is present at a concentration of about 0.1% to 5%, more preferably about 0.1% to 2.5%, even more preferably about 0.1% to 1%, most preferably about 0.2% to 0.5%. In a particularly preferred embodiment, the excipient, in particular the polyacrylic acid polymer, is present at a concentration of about 0.25% or 0.35%.

In certain embodiments, the pharmaceutical composition according to the invention further comprises a salt. It has been surprisingly shown in FIG. 7 that increasing the salt concentration in the medium increases the activity of the endolysin when formulated together with a polyacrylic acid polymer. The term “salt” as used herein, generally refers to ionic compounds comprised of both cations (positively charged ions) and anions (negatively charged ions) so that the product is electrically neutral (without a net charge). These component ions may be inorganic or organic ions, monatomic or polyatomic, monovalent or multivalent. Preferably, the salt comprised in the pharmaceutical composition according to the invention is a pharmaceutically acceptable salt. In a preferred embodiment, the salt comprised in the pharmaceutical composition according to the invention is sodium chloride (NaCl).

The salt, in particular the NaCl, may be present in the composition in any suitable concentration. In certain embodiments, the pharmaceutical composition according to the invention comprises NaCl at a concentration ranging from about 0% to about 12.5%, preferably from about 1% to about 10%, more preferably from about 2.5% to about 7.5%. In certain embodiments, the pharmaceutical composition according to the invention comprises NaCl at a concentration of about 0.5%, about 1%, about 1.5%, about 2%, about 2.5%, about 3%, about 3.5%, about 4%, about 4.5%, about 5%, about 5.5%, about 6%, about 6.5%, about 7%, about 7.5%, about 8%, about 8.5%, about 9%, about 9.5%, about 10%, about 10.5%, about 11%, about 11.5%, about 12%, or about 12.5%. In certain embodiments, the pharmaceutical composition according to the invention comprises NaCl at a concentration of about 0.5%, about 1%, about 1.5%, about 2%, about 2.5%, about 3%, about 3.5%, about 4%, about 4.5%, about 5%, about 5.5%, about 6%, about 6.5%, about 7%, about 7.5%, about 8%, about 8.5%, about 9%, about 9.5%, about 10%, about 10.5%, about 11%, about 11.5%, about 12%, or about 12.5%.

It is to be understood that the concentrations provided herein are to be understood as weight/volume percentage concentrations (w/v %) when the composition is formulated as a liquid, and as weight/weight percentage concentrations (w/w %) when the composition is formulated as a solid.

The pharmaceutical composition as provided herein may be employed as solids, such as tablets or filled capsules, or liquids such as solutions, suspensions, emulsions, elixirs, or capsules filled with the same, all for oral use, or in the form of a suppository for local (including intra-vaginal) use. Yet, also other means of administration are envisaged in context of this invention. Such means may comprise, inter alia, parenteral administration(s).

Also, pharmaceutical compositions for topical and/or local administration are envisaged, for example pharmaceutical compositions in form of (topical) gels, lotions or creams or via a pessary, i.e., a vaginal suppository. Such a pessary/vaginal suppository may be coated with the pharmaceutical composition described herein. The pharmaceutical compositions and unit dosage forms thereof may comprise ingredients in conventional proportions, with or without additional active compounds or principles, and such unit dosage forms may contain any suitable effective amount of the active ingredient commensurate with the intended daily dosage range to be employed. Compositions for uses of the invention may also be liquid formulations including, but not limited to, aqueous or oily suspensions, solutions, emulsions, syrups, and elixirs. The compositions may also be formulated as a dry product for reconstitution with water or other suitable vehicle before use. Such liquid preparations may contain additives including, but not limited to, suspending agents, emulsifying agents, non-aqueous vehicles and preservatives. Suspending agents include, but are not limited to, sorbitol syrup, methyl cellulose, glucose/sugar syrup, gelatin, hydroxyethylcellulose, carboxymethyl cellulose, aluminum stearate gel, and hydrogenated edible fats. Emulsifying agents include, but are not limited to, lecithin, sorbitan monooleate, and acacia. Nonaqueous vehicles include, but are not limited to, edible oils, almond oil, fractionated coconut oil, oily esters, propylene glycol, and ethyl alcohol. Preservatives include, but are not limited to, methyl or propyl p-hydroxybenzoate and sorbic acid. Further materials as well as processing techniques and the like are set out in Part 5 of Part 5 of Remington's “The Science and Practice of Pharmacy”, 22nd Edition, 2012, University of the Sciences in Philadelphia, Lippincott Williams & Wilkins.

Solid compositions of the invention may be in the form of tablets or lozenges formulated in a conventional manner. Tablets may be coated according to methods well known in the art. Injectable compositions are typically based upon injectable sterile saline or phosphate-buffered saline or other injectable carriers known in the art.

The pharmaceutical composition of the invention may also be formulated as suppositories, which may contain suppository bases including, but not limited to, cocoa butter or glycerides. Compositions of this invention may also be formulated transdermal formulations comprising aqueous or non-aqueous vehicles including, but not limited to, creams, ointments, lotions, pastes, medicated plaster, patch, or membrane. Compositions of the invention may also be formulated for parenteral administration including, but not limited to, by injection or continuous infusion. Formulations for injection may be in the form of suspensions, solutions, or emulsions in oily or aqueous vehicles, and may contain formulation agents including, but not limited to, suspending, stabilizing, and dispersing agents. The composition may also be provided in a powder form for reconstitution with a suitable vehicle including, but not limited to, sterile, pyrogen-free water.

The pharmaceutical composition of the invention may also be formulated as a depot preparation, which may be administered by implantation or by intramuscular injection. The compositions may be formulated with suitable polymeric or hydrophobic materials (as an emulsion in an acceptable oil, for example), ion exchange resins, or as sparingly soluble derivatives (as a sparingly soluble salt, for example).

The pharmaceutical composition of the invention can also be administered in sustained release forms or from sustained release drug delivery systems. A description of representative sustained release materials can also be found in Remington's “The Science and Practice of Pharmacy”.

The pharmaceutical composition according to the invention may further comprise one or more pharmaceutically acceptable additional ingredient(s) such as alum, stabilizers, antimicrobial agents, buffers, coloring agents, flavoring agents, adjuvants, and the like. It is preferred that the pharmaceutical composition for uses of the invention does not comprise imidazole.

The pharmaceutical composition according to the invention is preferably used for the treatment of bacterial vaginosis. It is thus preferred herein that the pharmaceutical composition according to the invention is formulated for intra-vaginal delivery. Accordingly, in a particular embodiment, the invention relates to the pharmaceutical composition according to the invention, wherein said composition is suitable for intra-vaginal delivery of the active agent. The skilled person is capable of formulating a pharmaceutical composition comprising the active agent and the excipient disclosed herein that is suitable for intravaginal delivery.

However, the pharmaceutical composition of the invention may also be used for the treatment of bacterial infection, in particular Gardnerella infections, in male subjects. In such embodiments, it is preferred that the pharmaceutical composition according to the invention is formulated for administration into or on the glans penis, prepuce or urethral entry. The skilled person is capable of formulating a pharmaceutical composition comprising the active agent and the excipient disclosed herein that is suitable for delivery into or on the glans penis, prepuce or urethral entry.

In a particular embodiment, the invention relates to the pharmaceutical composition according to the invention for use in treating a bacterial infection, preferably bacterial vaginosis.

That is, the pharmaceutical composition of the invention comprising the Gardnerella-specific polypeptide and the polyacrylic acid polymer can be used in the treatment of bacterial infections, in particular bacterial infections that are caused by bacteria of the genus Gardnerella. Accordingly, the pharmaceutical composition of the invention may be used for the treatment of infections caused, without limitation, by one or more of G. vaginalis sensu stricto, G. leopoldii, G. piotii, and/or G. swidsinskii.

Gardnerella species, such as G. vaginalis is the most common cause of bacterial vaginosis. As used herein “Bacterial Vaginosis” (BV), also been referred to in the literature as bacterial vaginitis, non-specific vaginosis and non-specific vaginitis refers to the most common vaginal infection worldwide. In one embodiment, BV is defined as a pathological state characterized by the loss of normal vagina flora, particularly of H₂O₂-producing species of Lactobacillus, and the simultaneous overgrowth of anaerobic bacteria, often including those from the genus Gardnerella. The genus Gardnerella contains at least 13 species, and the most frequent ones were renamed G. vaginalis sensu stricto, G. leopoldii, G. piotii, and G. swidsinskii (Vaneechoutte et al., 2019 Int. J. Syst. Evol. Biol. 898661). In one preferred embodiment of the therapeutic use of the invention, the BV to be treated is a bacterial infection characterized by the presence of at least one strain of the Gardnerella genus selected from the group consisting of Gardnerella vaginalis sensu strict, Gardnerella leopoldii, Gardnerella piotii and Gardnerella swidsinskii, and any other Gardnerella species. Thus, in one embodiment, the pharmaceutical composition according to the invention comprises a Gardnerella-specific endolysin, preferably a recombinant Gardnerella-specific endolysin, as described herein, for use in treating a bacterial vaginosis, wherein said bacterial vaginosis is characterized by the presence of infective bacteria of species Gardnerella vaginalis sensu stricto, Gardnerella leopoldii, Gardnerella piotii, Gardnerella swidsinskii, and/or any other species in the genus Gardnerella. It is understood herein that a bacterial vaginosis which “is characterized by the presence of” (or “is caused by” as used herein interchangeably) certain bacterial species in the genus Gardnerella refers to the overgrowth of said bacteria (also referred herein as “infective bacteria”) in the vagina microflora of a patient, leading to vaginal dysbiosis and/or the loss of Lactobacillus dominance. Methods to determine whether a bacterial vaginosis is characterized by the presence of infective bacteria of bacterial species in the genus Gardnerella are known to the skilled person. As an example, a PCR test that checks for presence of Gardnerella strains to diagnose BV can be used.

It is important to understand that Gardnerella infections can also affect men, where they can cause severe and malodourous inflammation and swelling of the inner foreskin and glans. Accordingly, the pharmaceutical composition of the present invention may also be used to in the treatment of Gardnerella infection in male subjects, in particular where the infection affects the glans penis, the prepuce or the urethral entry.

Accordingly, in a particular embodiment, the invention relates to the pharmaceutical composition for use according to the invention, which is to be administered locally into the vagina of a female subject and/or into or on the glans penis, prepuce or urethral entry of a male subject.

In a particular embodiment, the invention relates to the pharmaceutical composition according to the invention for use in treating bacterial vaginosis, wherein the polypeptide, in particular the endolysin, is to be administered to a patient who previously failed a treatment with antibiotics and/or who suffers from bacterial vaginosis wherein the infective bacteria are resistant to a treatment with antibiotics.

That is, in certain embodiments, the BV to be treated with the pharmaceutical composition described herein is characterized by the presence of a Gardnerella strain (i.e., a strain from the genus Gardnerella) which is resistant to one or more antibiotics. In one preferred embodiment, said one or more antibiotic(s) is/are selected from the group consisting of the nitroimidazoles and Clindamycin. In one even more preferred embodiment, said Gardnerella strain is resistant to Metronidazole, Tinidazole, Secnidazole, Clindamycin or any combination thereof. In one even more preferred embodiment, said Gardnerella strain is resistant to Metronidazole and/or Clindamycin. In one even more preferred embodiment, the said Gardnerella strain is resistant to Metronidazole. In one most preferred embodiment, said Gardnerella strain is resistant to highly resistant to Metronidazole.

As used herein, “a patient who previously failed a treatment with antibiotics” refers to a patient who has a history of bacterial vaginosis, i.e., who contracted (or already experienced) symptoms of bacterial vaginosis in the past, who has been treated for said bacterial vaginosis by antibiotics and who relapsed, i.e., for whom the symptoms reappeared. Such a patient can also be referred to as a patient suffering from recurrent BV. As used herein, the terms “a patient who has history with bacterial vaginosis”, “a patient who already experienced symptoms of bacterial vaginosis” and “a patient suffering from recurrent BV” can be used interchangeably and include, but are not limited to, patients who had one or more episodes of BV, preferably two or more episodes of BV, more preferably two or more episodes of BV in 6 months, even more preferably two or more episodes of BV within the last 6 months, or preferably three or more episodes of BV, more preferably three or more episodes of BV in 12 months, even more preferably three or more episodes of BV within the last 12 months.

Methods to diagnose BV in a patient or to prove episodes of BV are known to the skilled person in the art, e.g., BV can be diagnosed clinically by using clinical criteria (such as, e.g., the Amsel's diagnostic criteria) or microscopically, by determining the Nugent score from a vaginal Gram stain. Without being bound by any theory, relapses (or recurrences) of BV in patients after a treatment with antibiotics might be caused by the persistence of a residual infection due to the resistance of the infective bacteria to the antibiotics used. Accordingly, in one preferred embodiment of the invention, the patient suffers from a bacterial vaginosis wherein the infective bacteria are resistant to antibiotics treatment. Thus, in one preferred aspect of the invention, the Gardnerella-specific pharmaceutical composition described herein is for use in treating a bacterial vaginosis, wherein the pharmaceutical composition is to be administered to a patient who previously failed a treatment with antibiotics and who suffers from a bacterial vaginosis wherein the infective bacteria are resistant to antibiotics treatment. As defined herein below, the infective bacteria of the BV to be treated herein are more preferably highly resistant to antibiotics treatment. In one preferred embodiment of the therapeutic uses of the invention, the patient to be treated suffers from BV wherein the infective bacteria of said BV are resistant, preferably highly resistant, to a treatment with Metronidazole, Tinidazole, Secnidazole, Clindamycin or any combination thereof. In one more preferred embodiment of the therapeutic uses of the invention, the patient to be treated suffers from BV wherein the infective bacteria of said BV are resistant, preferably highly resistant, to a treatment with Metronidazole and/or Clindamycin. In one even more preferred embodiment of the therapeutic uses of the invention, the patient to be treated suffers from BV wherein the infective bacteria of said BV are resistant, preferably highly resistant, to a treatment with Metronidazole.

In the context of the present invention, “resistance” of bacterial strains (preferably Gardnerella strains) with regards to antibiotics refers to the ability of the strains to resist the activity of the antibiotic to which it was previously susceptible and permits them to survive the antibiotics treatment. Antibiotic resistance might occur naturally (“intrinsic resistance”) or might be induced by the misuse of antibiotics in humans and animals (“acquired resistance”). Methods how to determine resistance or susceptibility of bacterial strains to antibiotics are known to the skilled person. As an example, EUCAST breakpoints for Gram-positive anaerobes (v11, 2021) can be used. An alternative definition is given in Petrina et al. (2017, Anaerobe 47, 115-119), where slightly higher resistance breakpoints are used, because the topical formulations of some nitroimidazoles and Clindamycin can establish concentrations in the mg/ml range in vaginal fluid, much higher than what is achievable with orally delivered antibiotics. According to this alternative, more stringent definition (also used in the appended examples), Resistance (R) can be defined as a MIC value superior or equal to 32 μg/ml for Metronidazole and as a MIC value superior 8 μg/ml for Clindamycin, whereas Sensitivity(S) can be defined as a MIC value inferior or equal to 8 μg/ml for Metronidazole and inferior or equal to 2 μg/ml for Clindamycin. The terms “Minimum Inhibitory Concentration” and “MIC” are used herein interchangeably and refer to the lowest concentration of a chemical, usually a drug, which prevents visible growth of bacterium. MIC can be defined as the minimal concentration of antibiotic at which no growth was detectable after 48 h by OD measurement. Accordingly, in one preferred embodiment of the therapeutic uses of the invention, the patient to be treated suffers from BV wherein the infective bacteria are resistant to antibiotics treatment as defined by EUCAST breakpoints. Other generally accepted resistance criteria might be used instead of and/or besides the EUCAST definition or the alternative definition mentioned above (Petrina et al., 2017, Anaerobe 47, 115-119) to define the resistance of the infective bacteria to antibiotics treatment in the context of the present invention.

As will be known to the skilled person in the art, “resistance” and “susceptibility” might also be defined in terms of MBC and/or MBEC values. The terms “Minimum Bactericidal Concentration” or “MBC” refer to the lowest concentration of an antibacterial agent required to kill a particular bacterium. Usually, the MBC90 or MBC99.5 is measured, i.e., the antibiotic concentration killing 90% or 99.5%, respectively, of cells within a defined time. MBC can be defined as the minimal concentration fully eradicating a suspension of, e.g., 2.5×10⁷ CFU/ml. While MIC is the lowest concentration of an antibacterial agent necessary to inhibit visible growth, MBC is the minimum concentration of an antibacterial agent that results in bacterial death of all cells in suspension up to a defined limit of detection, resulting in killing of at least 90% (MBC90) or at least 99.5% (MBC99.5) of bacteria. The terms “Minimum Biofilm Eradicating Concentrations” or “MBEC” refer to the lowest concentration of an antibacterial agent required to reduce a population of bacteria growing as biofilm below the limit of detection.

Without being bound by any theory, the resistance of the infective bacteria to antibiotics might be involved in (or being partially or substantially responsible of) the relapses (or recurrences) of BV in patients suffering from recurrent BV. Therefore, a patient suffering from bacterial vaginosis wherein the infective bacteria are resistant to a treatment with antibiotics is likely to fail a treatment with antibiotics. Accordingly, in one preferred embodiment of the invention, the patient to be treated and who suffers from bacterial vaginosis wherein the infective bacteria are resistant to a treatment with antibiotics is a patient who is prone to fail a treatment with antibiotics. As used herein, a patient “who is prone to fail a treatment with antibiotics” refers to a patient who is at high risk of failing a treatment with antibiotics, i.e., for whom the likelihood of relapse (e.g., within 12 months) is very high if said patient were to be treated with antibiotics. Methods to determine whether a patient will be prone to fail a treatment with antibiotics are known to the skilled person. As an example, a clinical sample can be collected, and the resistance of the vaginal microflora strains assessed. Likewise, a patient suffering from bacterial vaginosis wherein the infective bacteria are resistant to a treatment with antibiotics is likely to have already (i.e., previously) failed a treatment with antibiotics (as defined above). Accordingly, in one further preferred embodiment of the invention, the patient to be treated and who suffers from bacterial vaginosis wherein the infective bacteria are resistant to a treatment with antibiotics is a patient who previously failed a treatment with antibiotics.

According to the above definition of resistance, “High Resistance” (HR) can be defined as a MIC value superior or equal to 256 μg/ml for Metronidazole and a MIC value superior to 64 μg/ml for Clindamycin, i.e., 8-fold higher than the resistance breakpoints of the alternative definition, which are themselves already higher than the standard resistance breakpoints of the EUCAST definition. Accordingly, in one embodiment of the therapeutic uses of the invention, the patient to be treated suffers from BV wherein the infective bacteria of said BV are resistant, preferably highly resistant, to antibiotics treatment, preferably as defined by EUCAST breakpoints. In one preferred embodiment of the therapeutic uses of the invention, the patient to be treated suffers from BV wherein the infective bacteria of said BV are resistant, preferably highly resistant, to a treatment with Metronidazole, Tinidazole, Secnidazole, Clindamycin or any combination thereof. In one more preferred embodiment of the therapeutic uses of the invention, the patient to be treated suffers from BV wherein the infective bacteria of said BV are resistant, preferably highly resistant, to a treatment with Metronidazole and/or Clindamycin. In one even more preferred embodiment of the therapeutic uses of the invention, the patient to be treated suffers from BV wherein the infective bacteria of said BV are resistant, preferably highly resistant, to a treatment with Metronidazole.

In certain embodiments, the pharmaceutical composition according to the invention is used in treating bacterial vaginosis, wherein said patient previously failed a treatment with antibiotics and/or wherein the infective bacteria of said bacterial vaginosis are resistant to antibiotics treatment.

As used herein, the terms “treatment with antibiotics” and “antibiotics treatment” are used interchangeably and preferably refer to a treatment with the antibiotics recommended or approved for the treatment of BV. Antibiotics that are currently recommended or approved for the treatment of BV include the Nitroimidazoles, including but not limited to Metronidazole, Tinidazole, and Secnidazole, and Clindamycin. Thus, in one embodiment, said “treatment with antibiotics” or “antibiotics treatment” is a treatment with a nitroimidazole and/or Clindamycin. In a preferred embodiment, the treatment with antibiotics described herein is a treatment with Metronidazole, Tinidazole, Secnidazole, Clindamycin or any combination thereof. In a more preferred embodiment, the antibiotics treatment described herein is a treatment with Metronidazole, and/or Clindamycin. In an even more preferred embodiment, the antibiotics treatment described herein is a treatment with Metronidazole.

As used herein, “treatment” and “treating” and the like generally mean obtaining a desired pharmacological and physiological effect. The effect may be prophylactic in terms of preventing or partially preventing a disease, symptom or condition thereof and/or may be therapeutic in terms of a partial or complete cure of a disease, condition, symptom or adverse effect attributed to the disease. The term “treating” as used herein covers any treatment of a bacterial vaginosis in a mammal, particularly a human, and includes: (a) preventing bacterial vaginosis from occurring in a patient which may be predisposed to bacterial vaginosis but has not yet been diagnosed as having it; (b) inhibiting bacterial vaginosis, i.e., arresting its development; or relieving the bacterial infection, i.e., causing regression of the bacterial infection and/or its symptoms or conditions such as improvement or remediation of damage. In particular, treatment of bacterial vaginosis comprises preventing, decreasing or even eradicating the infection, for instance by killing the infective bacteria and, thus, controlling, reducing or inhibiting bacterial proliferation as well as reducing the number of viable bacterial cells. Herein it is preferred that the disease, i.e., BV, is treated therapeutically in terms of a partial or complete cure of the disease or the symptoms.

It will be appreciated by persons skilled in the art that the active agent to be used according to the invention is generally administered in admixture with a suitable pharmaceutical excipient, diluent or carrier selected with regard to the intended route of administration and standard pharmaceutical practice (for example, see Remington: The Science and Practice of Pharmacy, 19th edition, 1995, Ed. Alfonso Gennaro, Mack Publishing Company, Pennsylvania, USA). For example, the active agent can be administered locally, i.e., locally into the vagina of a female subject and/or, in a male subject into or on the glans penis, prepuce or urethral entry.

Herein the term “(administration) into or on the glans penis” also includes “(administration) into and on the glans penis”. In line with this, the term “(administration) into or on the glans penis, prepuce or urethral entry of a male subject” also includes “(administration) into and on the glans penis and on the prepuce and on the urethral entry of a male subject”. In another embodiment, the active agent can be co-administered with a compound or composition which adjusts the pH of the vagina. In some embodiment the compound or composition adjusts the pH of the vagina to pH 4.0 to 6.0, preferably to pH 5.0.

The terms “patient” and “subjects” are used herein interchangeably and refer to mammals. For examples, mammals contemplated by the present invention include human, primates, domesticated animals such as cattle, sheep, pigs, horses, laboratory rodents and the like. It is preferred that the patient is a human being. It is even more preferred that the patient is a woman (also referred herein as a “female subject”).

It will be appreciated that the pharmaceutical compositions for uses as described herein may be administered to a subject in combination with one or more additional therapeutic agents. For example, the endolysins and pharmaceutical compositions described herein may be administered to a subject in combination with:
(a) one or more conventional antibiotic treatments. Such antibiotics may include Clindamycin, Metronidazole or any other suitable antibiotics known by a skilled person in the art;
(b) one or more additional endolysins, or nucleic acid molecules, vectors, host cell or bacteriophage capable of expressing the same;
(c) a compound or composition which adjusts the pH of the vagina, preferably to pH 4.0 to 6.0, more preferably to about pH 5.0. Such pH adjusting compounds may include phosphate, lactic acid (e.g., the natural acidification substance which Lactobacilli secrete to establish an acidic milieu) or other organic acids, e.g., carboxy-substituted polymers;
(d) a therapy to neutralize the toxins released upon bacterial lysis of G. vaginalis cells within the vagina. Suitable neutralizing therapies may include antibodies (see Babcock et al., 2006, Infect. Immun. 74:6339-6347) and toxin-absorbing agents such as tolevamer (see Barker et al., 2006, Aliment. Pharmacol. Ther. 24:1525-1534);
(e) a probiotic.

In one embodiment of the uses of the invention, a host cell or pharmacological composition comprising a host cell is used to deliver the endolysin (preferably a host cell).

In a particular embodiment, the invention relates to a use of the pharmaceutical composition according to the invention for the manufacture of a medicament for the treatment or the prevention of a bacterial infection, preferably bacterial vaginosis.

In a particular embodiment, the invention relates to a method of treating or preventing a bacterial infection, preferably bacterial vaginosis, in a subject in need thereof comprising administering to said subject a therapeutically effective amount of the pharmaceutical composition according to the invention.

Also provided by the present invention is a method of treating a bacterial infection, in particular bacterial vaginosis, as described herein above, wherein the method comprises administering to said patient a therapeutically effective amount of a pharmaceutical composition comprising a Gardnerella-specific polypeptide, preferably a Gardnerella-specific endolysin, and a polyacrylic acid polymer.

Embodiments and definitions described herein above for the therapeutic uses according to the invention apply in the context of the method of treating BV mutatis mutandis.

One aspect of the invention relates to a composition comprising a polyacrylic acid polymer for use in the treatment and/or prevention of bacterial infections, wherein the polyacrylic acid polymer reduces biofilms.

In another aspect, the invention relates to a non-therapeutic use of a polyacrylic acid polymer in reducing bacterial biofilms.

That is, the inventors surprisingly found that polyacrylic acid polymers can reduce Gardnerella biofilms, even in the absence of a Gardnerella-specific polypeptide; see FIG. 2 Panel A and Panel B. In contrast, polyacrylic acid polymers have no killing activity against planktonic Gardnerella; see FIG. 5. Thus, it was shown that polyacrylic acid polymers specifically act directly against bacterial biofilms. Based on these findings, polyacrylic acid polymers may be used as components of pharmaceutical compositions for the treatment and/or prevention of bacterial infections, in particular when the bacterial infections involve the formation of biofilms.

A polyacrylic acid polymer is defined reduce or dissolve biofilms, if contacting the polyacrylic acid polymer with a biofilm reduces the number of viable bacteria (CFU/mL) in the biofilm by a factor of at least 2, at least 3, at least 4, at least 5, at least 6, at least 7, at least 8, at least 9, or at least 10.

For that, biofilms (e.g., of G. vaginalis ATCC 14018) may be generated in sBHIG (in g/L: Brain Heart Infusion: 37, gelatine: 20, yeast extract: 5, starch: 1, glucose: 2.5; water: up to 1000 mL) pH 7 for 48 hours on pre-coated tissue culture plates. The resulting biofilms may then be treated in sBHIG pH 4.5 for 24 hours with PAA. Alternatively, the biofilms may be generated in sNYC (in g/L: HEPES: 2.4, proteose peptone: 15, yeast extract: 3.8, NaCl: 5, glucose: 10; water: up to 1000 mL) pH 7 for 48 h, and may then be treated with PAA in sNYC pH 6 for 24 h. For the biofilm treatment with PAA, a PAA master stock (27% (w/v)) may be used to prepare a 0.35% PAA (v/v) working stock. After incubation, the biofilm may be washed and dislodged by vigorous pipetting. A serial dilution may be done in PBS (1×PBS-DPBS, no calcium, no magnesium, Thermo Scientific, cat. no. 14190169) and spotted on Chocolate Agar plates (BD, cat. no. 254060) to count the number of viable cells (CFU/ml).

In certain embodiments, the bacterial biofilm is a mucosal biofilm. In certain embodiments the (mucosal) biofilm is formed by bacteria of the genus Gardnerella.

In certain embodiments, the bacterial infection is bacterial vaginosis, as defined herein above.

Preferably the pharmaceutical composition further comprises a polypeptide having a killing activity against Gardnerella, as described herein. That is, it has been demonstrated in FIG. 2 that polyacrylic acid acts synergistically with the endolysin PM-477 against Gardnerella biofilms.

The polyacrylic acid polymer comprised in the pharmaceutical composition according to the invention may be a crosslinked polyacrylic acid polymer or a carbomer. The definitions of the polyacrylic acid polymer herein above apply mutatis mutandis.

The term “about”, as used herein, refers to a numeric value, including, for example, whole numbers, fractions, and percentages, whether or not explicitly indicated. The term “about” generally refers to a range of numerical values (e.g., +/−1-3% of the recited value) that one of ordinary skill in the art would consider equivalent to the recited value (e.g., having the same function or result). In some instances, the term “about” may include numerical values that are rounded to the nearest significant figure.

The present invention is further described by reference to the following non-limiting figures and examples.

### BRIEF DESCRIPTION OF THE DRAWINGS

FIGS. 1A and 1B: FIG. 1A, Kinetic study of PM-477 against G. vaginalis Gv9 (ATCC 14018T) shows a strong lytic effect which is time and dose dependent. Suspensions of Gardnerella cells were treated with recombinantly expressed PM-477 for 1 h, 5 h and 24 h at 2.5, 10, and 40 μg/ml and the colony forming units (CFU) per ml was determined in a logarithmic scale and compared to the buffer treated control. FIG. 1B, Suspensions of other vaginal BV pathogens (in grey) as well as probiotic Lactobacilli (in white boxes) were treated with PM-477 for 5 h at 10, and 100 g/ml and the colony forming units (CFU) per ml was determined in a logarithmic scale and compared to the buffer treated control. LOD, indicates the limit of detection. Statistically significance (One-way ANOVA, comparison of control to 10 and 10 g/mL PM-477 treatment for each strain) is indicated; ***, P<0.001, **, P<0.01, *, P<0.05.

FIG. 2: FIG. 2, Panel A, Gardnerella biofilms (using the type strain G. vaginalis ATCC 14018) were treated with 8 μg/ml PM-477 (and thus below the concentration that is typically used to eradicate Gardnerella biofilms), 0.35% (w/v) PAA and the combination thereof for 24 hours at pH 4.5 in sBHIG medium. FIG. 2, Panel B, Gardnerella biofilms (G. vaginalis ATCC 14018) were treated with 32 μg/ml H2B10B11, 0.25% (w/v) PAA and the combination thereof for 24 hours at pH 6 in sNYC medium.

Cells were dislodged by pipetting and quantitative plating was used to determine the remaining viable cells (CFU/ml). The bactericidal effect is visualized by log₁₀ reductions in CFU/ml compared to the buffer treated control. The grey column indicates the theoretical additional effect of the mean log reduction of PM-477/H2B10B11 and PAA. Mean values with standard deviation are shown.

FIGS. 3A-3C: Lytic activity of two PM-477 formulations on six BV-associated pathogens and one commensal Lactobacillus species. Sensitivity of the strains was tested by treating fresh liquid culture suspensions of the strains for 5 hours with a no-EL control, PM-477 formulated with PAA to pH 4.5 (FIG. 3A), or PM-477 formulated with PAA to a pH of 5.0 (FIG. 3B). Viability after the indicated treatment was determined by quantitative spotting (FIGS. 3A & 3B) and viability reduction for both formulations (FIG. 3C) was calculated by subtracting the log 10 CFU/mL in replicates of the formulation treatments from the median of the respective unamended control. Shown are triplicates, range, and median. LOD: limit of detection. Points below the LOD indicate we reached the limit of detection.

FIG. 4: Influence of PAA concentration of the viability of planktonic Gv9 cells. Sensitivity to PAA was tested by treating fresh liquid culture suspensions of Gv9 with pH 5 sNYCB with varying concentrations of PAA for 90 minutes at 37° C. Viability after treatment was determined by quantitative spotting. Shown are triplicates and range with a horizontal line at the median. PAA: poly(acrylic acid). LOD: limit of detection.

FIG. 5: Influence of PAA on the viability of five BV-associated pathogens and one commensal. Sensitivity of the strains to PAA was tested by treating fresh liquid culture suspensions with NYCB medium at pH 5 with concentrations of PAA varying from 0.01% to 1% for 90 minutes and comparing this to untreated controls. Viability after treatment was determined by quantitative spotting. Data are depicted in triplicate, with a line at the median. PAA: poly-acrylic acid. LOD: limit of detection. Points below the LOD indicate we reached the limit of detection.

FIG. 6: Lytic activity of H2B10B11 formulated with Carbopol 974P on BV-associated pathogens and one commensal. Sensitivity of the strains was tested by treating fresh liquid culture suspensions of the strains for 20.5 h with a no-EL control or with a low concentration of H2B10B11 (2 μg/mL) formulated with 0.25% (w/v) CP 974P in medium adjusted to pH 5. Viability after the indicated treatment was determined by quantitative spotting. Shown are triplicates and median. LOD: limit of detection. Points below the LOD indicate that the limit of detection was reached.

FIG. 7: Activity of H2B10B11 formulated with Carbopol is tunable by modulating the salt content. A 90 min lytic potency assay on Gardnerella vaginalis was performed with 10 μg/mL H2B10B11 and/or 0.1% (w/v) Carbopol in medium adjusted to pH 5.5 with normal (86 mM, circle) and elevated salt concentration (345 mM, diamond). Potency of H2B10B11 was assessed via quantitative spotting. Triplicates with median and range are shown.

FIG. 8: Reduction of L. iners in swab samples of BV-positive women after treatment for 19 h. Thirty vaginal swab samples were treated overnight with 50 μg/mL H2B10B11 (black) or with 50 μg/mL H2B10B11 formulated with 0.1% (w/v) CP 974P and 1% NaCl (grey). The viable L. iners load was determined via viability qPCR using a L. iners-specific primer pair. DNA copies/μL in treated samples subtracted by the load in respective untreated baseline samples are plotted over the load in baseline samples. Linear regression across samples was performed, the slope is stated in the graph.

### EXAMPLES

### Example 1—PM-477 Endolysin is a Highly Active Antibacterial Enzyme that Targets the Genus Gardnerella and to a Minor Extent Lactobacillus iners

Bacterial vaginosis (BV) is characterized by an imbalance of the vaginal microbiome and a characteristic biofilm formed on the vaginal epithelium, which is initiated and dominated by Gardnerella bacteria. The inventors previously disclosed synthetic endolysins, originated from the type 1,4-beta-N-acetylmuramidase encoded on Gardnerella prophages (WO 2020/225335). These endolysins efficiently kill Gardnerella bacteria and to a minor extent also L. iners, another opportunistic pathogen in BV. Importantly, probiotic Lactobacilli such as L. crispatus and L. gasseri are not harmed by the treatment of PM-477. The inventors tested the killing activity of a representative endolysin (PM-477 (H₂B10)) on Gardnerella vaginalis.

Gardnerella bacteria in suspension (OD-0.1) were treated with the endolysin PM-477 (stock solution 700 μg/ml PM-477 in 50 mM MES pH 5.5, 200 mM NaCl, 8 mM MgSO₄) or with a buffer control and incubated anaerobically for 1, 5, and 24 hours. After the incubation period, the suspension was serially diluted, spotted on Chocolate agar plates, and the CFU/ml were counted. The efficacy was clearly time and dose dependent and already low concentrations of 10 and 40 μg/ml after 5 hours incubation reduced the cell number to the limit of detection (LOD). No regrowth was observed after 24 h. Interestingly, no further killing was recorded between 5 h and 24 h when treated with very low concentration of 2.5 μg/ml, indicating that an equilibrium may have formed. The inventors also tested the activity of PM-477 on other vaginal opportunistic pathogens as well as probiotic vaginal Lactobacilli. PM-477 proved to be specific for Gardnerella bacteria without effecting beneficial Lactobacilli or other opportunistic BV pathogens. The only exception was L. iners, where high concentrations of 100 μg/ml after 5 hours reduced the number of bacteria by 2.5 log 10 units. L. iners clearly differs from other Lactobacilli regarding cell wall constitution and metabolism and is considered as a pathogen in BV.

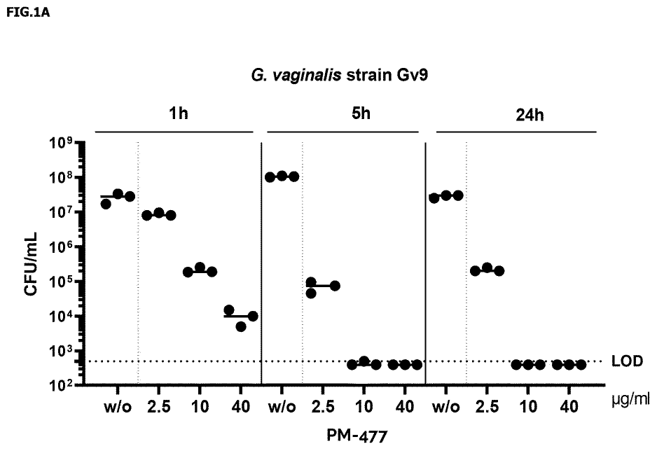

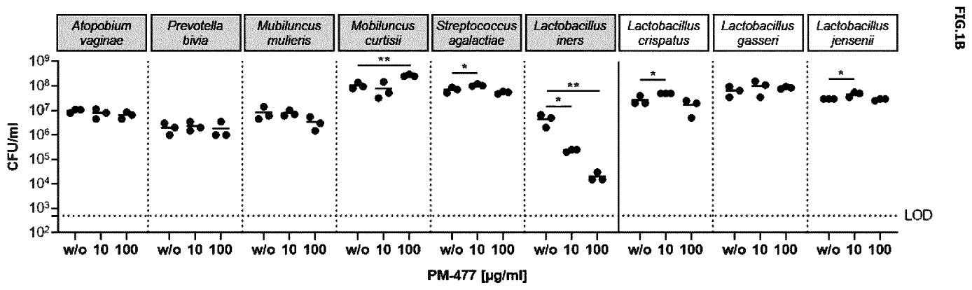

In summary, a strong bactericidal effect against Gardnerella was demonstrated for the recombinantly expressed endolysin PM-477 (H2B10) without harming L. crispatus, L. gasseri and L. jensenii, which are the most prevalent species in a healthy vaginal microbiome (see FIGS. 1A and 1B).

### Example 2—PAA as an Excipient Enhances the Killing Effect of Endolysins on Gardnerella Bacteria Growing as Biofilm

The inventors tested PAA (Sigma Aldrich, catalog number 323667-100G) as a vehicle for the Gardnerella-specific endolysins for the use of intravaginal application. The viscosity and mucoadhesive property of PAA guarantee a slow release of the active pharmaceutical ingredient (the endolysin) on the mucus layer in the vagina and its acidifying effect helps to restore the healthy vaginal microbiome. Unexpectedly, the presence of PAA also enhanced the potency of endolysins and Gardnerella bacteria were killed more efficiently. The inventors tested the Gardnerella-specific endolysins PM-477 and H₂B10B11 formulated with PAA on in vitro mono-species Gardnerella biofilms. The biofilms (G. vaginalis ATCC 14018) were generated in sBHIG (in g/L: Brain Heart Infusion: 37, gelatine: 20, yeast extract: 5, starch: 1, glucose: 2.5; water: up to 1000 mL) pH 7 for 48 hours on pre-coated tissue culture plates, then treated in sBHIG pH 4.5 for 24 hours with low concentrations of the endolysin PM-477 together with PAA, as well as PM-477 and PAA alone. Alternatively, the biofilms were generated in sNYC (in g/L: HEPES: 2.4, proteose peptone: 15, yeast extract: 3.8, NaCl: 5, glucose: 10; water: up to 1000 mL) pH 7 for 48 h, and were then treated with H2B10B11 or PAA or a combination of both in sNYC pH 6 for 24 h.

For the biofilm treatment with PAA and PM-477, a PAA master stock (27% (w/v)) was used to prepare a 0.35% PAA (v/v) working stock, by dilution with sBHIG pH 5, containing 8 μg/ml PM-477 (PM-477 master stock of 700 μg/ml was used for dilutions). In addition, the final mixture of PM-477 with the excipient PAA used for the biofilm treatment had pH 4.5 due to the buffering effect of 0.35% PAA. For the biofilm treatment with PAA and H₂B10B11, a PAA master stock (14.5% (w/v), adjusted to pH 6) was used to prepare a treatment solution containing 0.25% PAA and 32 μg/mL H₂B10B11 in sNYC pH 6. After incubation, the biofilm was washed and dislodged by vigorous pipetting. A serial dilution was done in PBS (1×PBS-DPBS, no calcium, no magnesium, Thermo Scientific, cat. no. 14190169) and spotted on Chocolate Agar plates (BD, cat. no. 254060) to count the number of viable cells (CFU/ml). For the tested strains, the Gardnerella biofilm eradication concentration of PM-477 was between 2 and 32 μg/ml. The concentration chosen herein was 8 μg/ml, and thus at the lower end of the range of the minimum biofilm eradication concentration (MBEC) for Gardnerella strains, to be able to see a potential synergistic effect of the excipient PAA and PM-477. It is to be noted that the MBEC is influenced by the medium and pH used for the experiment. Since the biofilm experiment with H₂B10B11 was carried out at pH 6, 32 μg/mL H2B10B11 were not sufficient to eradicate the biofilm in the absence of PAA.

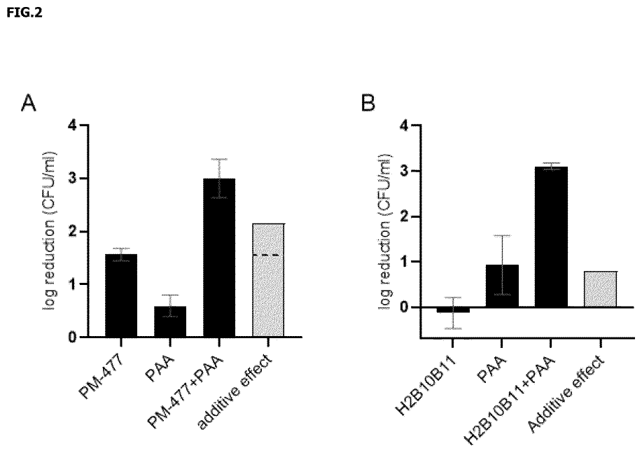

PM-477 (8 μg/ml) alone reduced the CFU/ml by 1.6 log₁₀ units, PAA (0.35% w/v) alone by 0.6 log₁₀ units, while the combination of PM-477 and PAA resulted in a clearly synergistic effect with killing rates of 3 log₁₀ units. The difference to the potential additive effect (0.6 log₁₀ units of PAA and 1.6 log₁₀ units of PM-477=2.2 log₁₀ unit reduction) was 0.8 log units (3 log₁₀ units in total) which translates into a 6-fold increased activity on a linear scale (FIG. 2, Panel A).

No relevant effect was observed with H2B10B11 (32 μg/mL) alone in medium at pH 6. PAA (0.25% w/v) alone reduced the CFU/mL by 0.9 log₁₀ units, while the combination of H₂B10B11 and PAA resulted in an obvious synergistic effect with a log₁₀ reduction of 3.1 (FIG. 2, Panel B).

### Example 3—PAA as an Excipient Enhances the Killing Effect of Endolysins on Gardnerella Bacteria and Other BV Pathogens Growing in the Planktonic Form

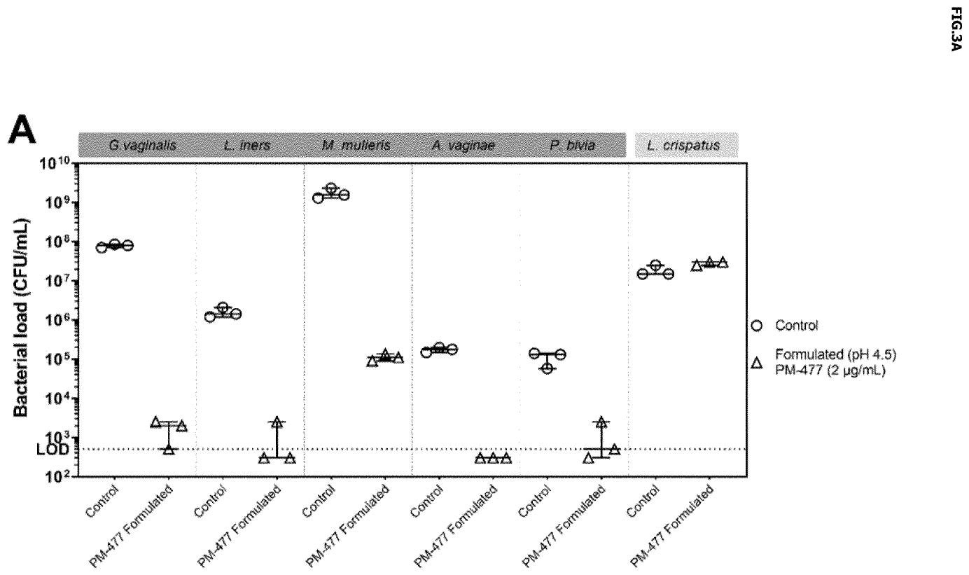

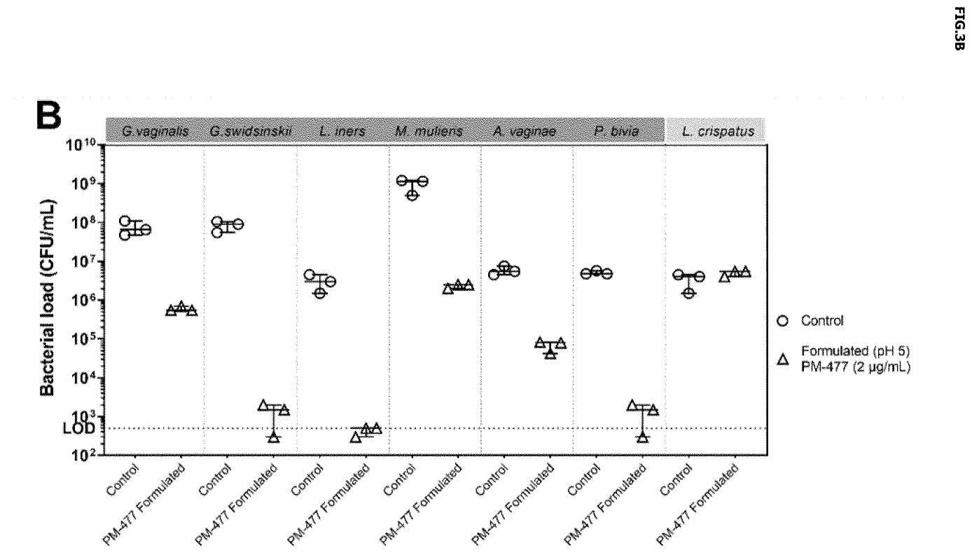

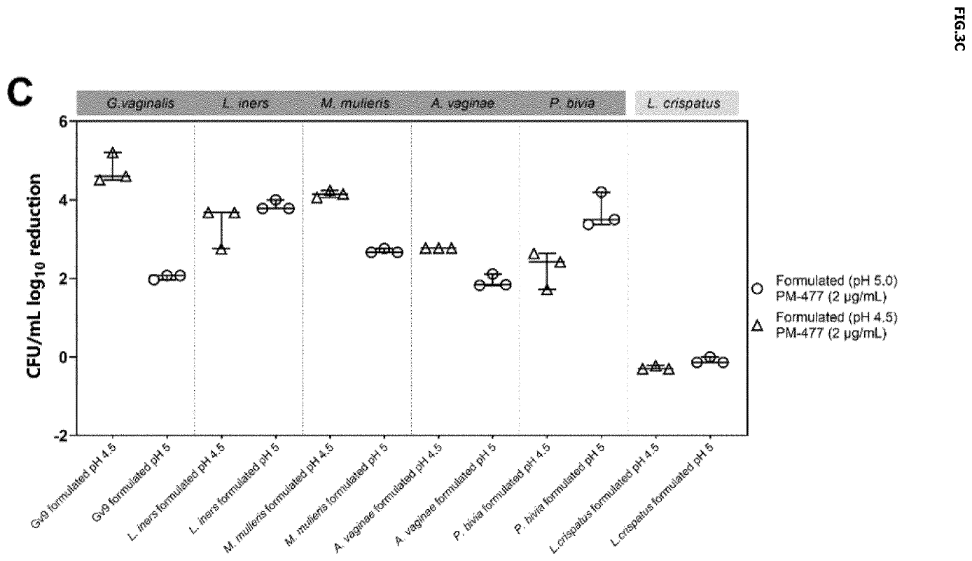

Next, the potency of two different formulations of PM-477 with 0.25% PAA (w/v; Sigma Aldrich, catalog number 323667-100G) as an excipient was tested against six BV associated pathogens and one commensal Lactobacillus species in a time-kill assay (FIGS. 3A-3C). Both formulations included 2 μg/mL PM-477 and 0.25% PAA and were adjusted to either pH 4.5 (FIG. 3A) or pH 5 (FIG. 3B) to simulate different PAA types and composition. Briefly, 4 g of PAA was added to 10 mL of dH₂O and heated to 50° C. while mixing on a heated stir plate. The pH of PAA solutions was adjusted to either 4.5 or pH 5 using 5 M NaOH. Then, the volumes were adjusted to 27.5 mL (corresponding to 14.5% PAA) and sterile filtered using a 0.22 μm syringe filter. These stocks were diluted to a final concentration of 0.25% PAA in glucose-supplemented New York City Broth (sNYCB) adjusted to the respective pH. Supplemented NYCB consists of 10 mM HEPES (Sigma Aldrich), 15 g/L Proteose Peptone (Sigma Aldrich), 3.8 g/L yeast extract (Thermo Fisher Scientific), 86 mM sodium chloride (Carl Roth), and 10 g/L α-D-glucose (Sigma Aldrich)).

Bacteria were grown on Chocolate Agar plates anaerobically for 48 h at 37° C., then scraped from the plates, and suspensions of 10⁸ CFU/ml were prepared for the experiment. For the time-kill experiments, the bacterial suspensions were mixed with the respective treatment stock solutions and incubated anaerobically for 5 h at 37° C. Then, the surviving bacteria were quantified by spotting 2 μL of a 10-fold dilution series (lowest dilution 10⁻⁵) on Chocolate Agar plates after anaerobic incubation at 37° C. for 48 h.

Both formulations were highly effective in reducing the bacterial load of all six pathogens but had no negative effect on the commensal L. crispatus (FIGS. 3A-3C). The formulation set to a slightly more acidic pH (4.5) reduced viable Gv9 (G. vaginalis ATCC 14018), L. iners, M. mulieris, by >3 log₁₀ and A. vaginae, and P. bivia by >2 log₁₀ when compared to the controls (FIG. 3C). When formulated to a less acidic pH (5.0), 2 g/mL PM-477 reduced Gv24 (G. swidsinskii GS 9838-1), L. iners, and P. bivia by >3 log₁₀ and Gv9, M. mulieris, and A. vaginae by >2 log₁₀ when compared to the controls without having any negative affect on the commensal L. crispatus (FIG. 3C).

In summary, formulation with PAA surprisingly extends the spectrum of activity of PM-477 to multiple BV-associated pathogens other than Gardnerella.

### Example 4—PAA Alone has No Killing Effect on Gardnerella and a Weak Killing Effect on Some Other BV Pathogens, but Only at Higher Concentrations

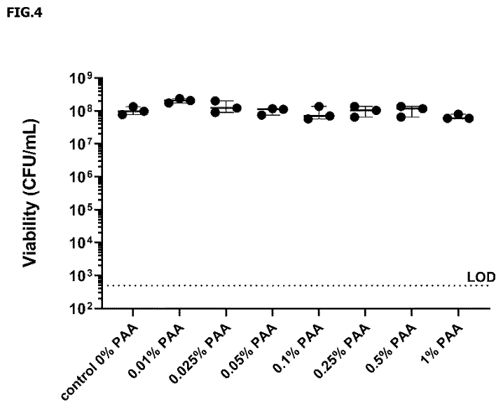

Next, the effect of PAA (Sigma Aldrich, catalog number 323667-100G) alone on Gardnerella and other BV-associated pathogens was assessed. As described in the previous Examples, bacterial suspensions were adjusted to 10⁸ CFU/ml, mixed with treatment stock solutions and incubated anaerobically for 90 minutes at 37° C., after which surviving bacteria were quantified by spotting of a 10-fold dilution series of the reactions. The spotting plates were incubated for 2-3 d prior to determination of CFU/mL. As shown in FIG. 4, PAA concentrations up to 1% did not reduce the viability of Gardnerella after incubation for 90 min.

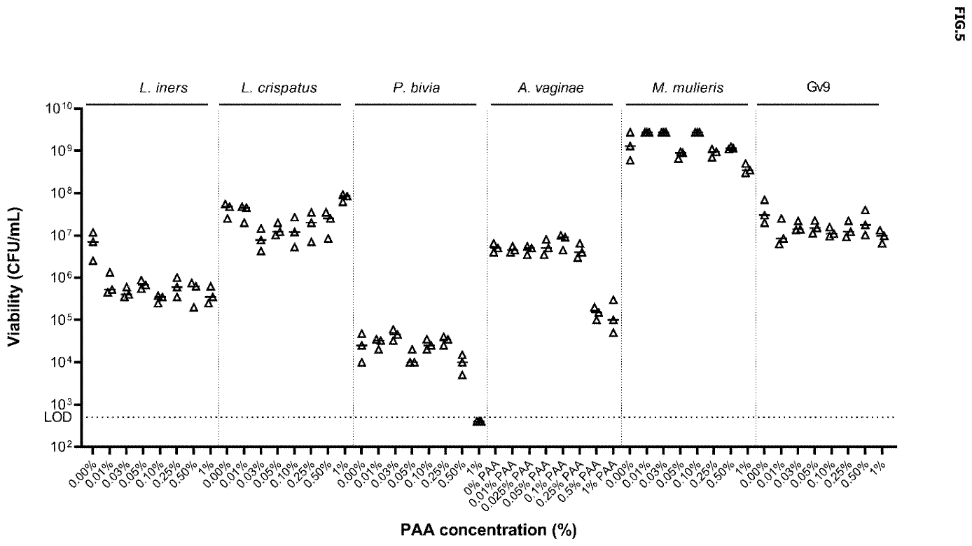

The sensitivity of five other BV associated pathogens and one commensal Lactobacillus species to PAA was also tested using the same assay. The four BV associated pathogens tested were L. iners, P. bivia, A. vaginae, and M. mulieris, while L. crispatus was used as the commensal (FIG. 5). The inventors tested concentrations of PAA ranging from 0.01% to 1% set to pH 5 on all the five organisms using 90 minute potency assays. PAA concentrations up to 1% had minimal influence (<0.6 log₁₀ decrease) on the viability of M. mulieris and L. crispatus. Viability of P. bivia, and A. vaginae decreased by 1.8 and 1.7 log₁₀ only in response to high concentrations of PAA (>0.5%), with lower concentrations having no effect (FIG. 5). Viability of L. iners decreased by ~1.1 log 10 in response to the lowest tested concentrations of PAA (0.01%) and did not decrease further with higher PAA concentrations (FIG. 5).

### Example 5—Carbopol 974P as an Excipient Enhances the Killing Effect of Endolysins on Planktonic BV-Associated Pathogens but not on the Commensal Lactobacillus Species

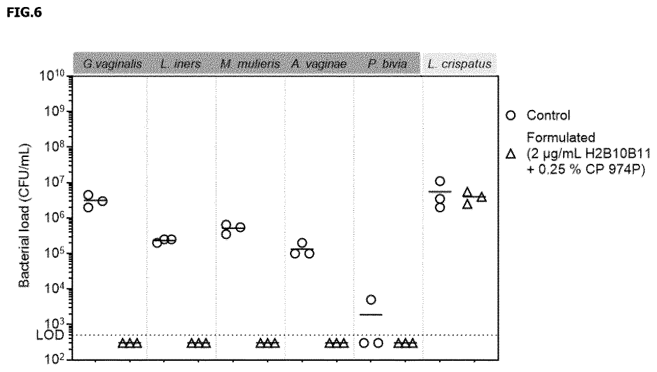

The potency of H2B10B11 formulated with 0.25% (w/v) Carbopol 974P NF Polymer (CP 974; The Lubrizol Corporation) was tested against five BV-associated pathogens and one commensal Lactobacillus species in a planktonic setting for 5 h in sNYC medium at pH 5 (FIG. 6). The formulation comprised 2 μg/mL H2B10B11 and 0.25% CP 974P with a final pH of 5. To yield the formulated treatment solution, 50 mg CP 974P were added to 15 mL sNYC pH 5. After mixing, the pH was re-adjusted to pH 5 using 5 M NaOH, and the volume adjusted to 20 mL with sNYC pH 5. H2B10B11 was added to a final concentration of 2 μg/mL from a 1.343 mg/mL stock in MES pH 5.5 buffer.

Gardnerella was grown on a Chocolate Agar plate, the other bacterial strains on Schaedler Agar plates with vitamin K1 and 5% sheep blood (BD, catalog number 254042) anaerobically for 48 h at 37° C., then scraped from the plates, and suspensions of 10⁸ CFU/ml were prepared for the experiment. The bacterial suspensions were mixed with either sNYC pH 5 or with sNYC containing formulated H2B10B11 pH 5 and incubated anaerobically for 20.5 h at 37° C. Then, the surviving bacteria were quantified by spotting 2 μL of a 10-fold dilution series (lowest dilution 10⁻⁵) on Chocolate/Schaedler Agar plates and anaerobic incubation at 37° C. for 48 h.

Formulated H2B10B11 eradicated the BV-associated pathogens G. vaginalis, L. iners, M. mulieris, A. vaginae, and P. bivia (FIG. 6). This is an unexpected observation, since the endolysin alone is not effective on non-Gardnerella strains except for to a smaller extend on L. iners (see FIG. 1B). Since the results obtained here with Carbopol 974P are comparable to the results presented in Example 3 (FIGS. 3A-3C), in which the formulation comprised PAA, it can be concluded that poly(acrylic acid) and its cross-linked pharmaceutical-grade form (carbomer) lead to a synergistic extension of lytic activity of the endolysin. Furthermore, likewise to PM-477 formulated with PAA, H2B10B11 formulated with CP 974P is not harming the beneficial L. crispatus (FIG. 6).

### Example 6—Activity of H2B10B11 Formulated with Carbopol is Tunable by Modulating the Salt Content

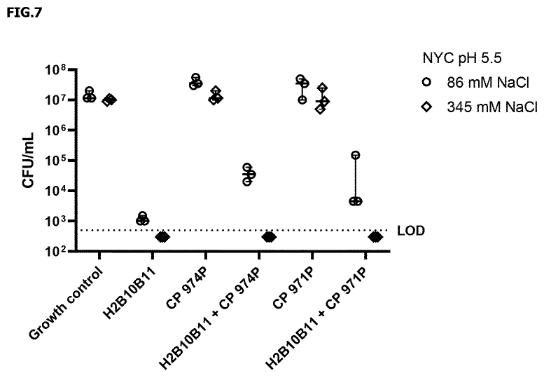

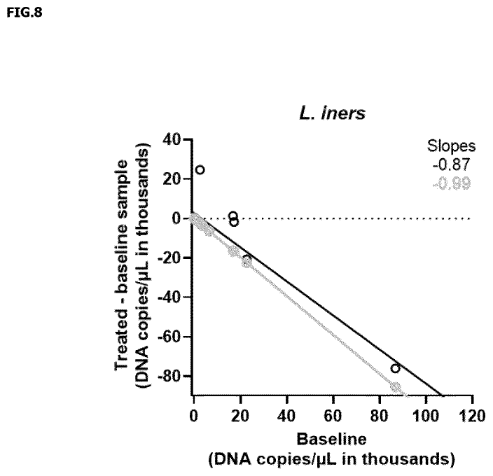

The potency of H2B10B11 alone or formulated with Carbopol 974P NF Polymer or Carbopol 971P NF Polymer (The Lubrizol Corporation) was tested against Gardnerella vaginalis in a planktonic setting for 90 min in NYC medium at pH 5.5 with normal (86 mM) or elevated (345 mM) NaCl concentration (FIG. 7). The formulation comprised 10 μg/mL H₂B10B11 and 0.1% Carbopol with a final pH of 5.5. To yield the formulated treatment solution, 40 mg CP 974P or CP 971P were added to 40 mL NYC with normal or elevated NaCl concentration. After mixing, the pH was adjusted to pH 5.5 using 5 M NaOH. H₂B10B11 was added to a final concentration of 10 μg/mL from a 1.345 mg/mL stock in MES pH 5.5 buffer.

Gardnerella was grown on a Chocolate Agar plate anaerobically for 48 h at 37° C., then scraped from the plates using PBS. The bacterial suspensions were mixed with the respective medium containing H₂B10B11, Carbopol or a combination of both to yield a final bacterial OD of 0.1. The reaction plate was incubated anaerobically for 90 min at 37° C. Then, the surviving bacteria were quantified by spotting 2 μL of a 10-fold dilution series (lowest dilution 10⁻⁵) on Chocolate Agar plates and anaerobic incubation at 37° C. for 48 h.

### Example 7—Reduction of L. iners in Vaginal Swab Samples of BV-Positive Women after Treatment for 19 h

Thirty vaginal swab samples from BV-positive women were treated for 19 h with 50 μg/mL H2B10B11 or with 50 μg/mL H2B10B11 formulated with 0.1% (w/v) CP 974P and 1% NaCl. For this, two vaginal swabs in Amies medium (Copan 480CE) were pooled, 400 μL NYC pH 5.5+10% horse serum was added, mixed thoroughly and frozen. Treatment reactions were performed in a 96-well plate using 114 μL of the sample and the treatment solution in MES buffer pH 5.5. Reaction plates were statically incubated for 19 h at 37° C. under anerobic conditions. Baseline (untreated samples) and treated samples were treated with PMAxx (Biotium), which intercalates into the DNA of dead cells and thereby minimizes any qPCR signal from dead cells. Briefly, 45 μL of 50 μM PMAxx premix I was added to the cell pellets, incubated on ice for 15 min and exposed to light in a PMA-Lite LED Photolysis Device for 15 min. PMAxx treatment was repeated twice with 2.25 μL of 1 mM PMAxx premix II. Tubes were centrifuged for 8 min at 8000 rpm, supernatants were discarded. Cell pellets were washed with saline, centrifuged for 10 min at 8000 rpm. Genomic DNA was extracted using the DNeasy 96 Blood & Tissue Kit.

The viable L. iners load was determined via viability qPCR using a L. iners-specific primer pair. qPCR was performed in a LightCycler 480 (Roche) using EvaGreen® qPCR Supermix (Solis Biodyne) with 2 μK of extracted DNA per reaction in a total volume of 10 μL. PCR conditions were as follows: initial denaturation at 95° C. for 12 min, followed by 40 cycles of 15 s at 95° C., 40 s at 57° C. and 30 s at 72° C., followed by melting analysis between 55 and 95° C. with a ramp rate of 2.5° C./s.

DNA copies/μL in treated samples subtracted by the load in respective untreated baseline samples are plotted over the load in baseline samples. Linear regression across samples was performed, the slope is stated in the graph. The slope reflects the overall reduction of L. iners across patient samples, i.e. 99% reduction with formulated H2B10B11.

1. A pharmaceutical composition comprising an effective amount of an active agent and a pharmaceutical acceptable excipient, wherein said active agent is a polypeptide having a killing activity against Gardnerella and wherein said excipient is a polyacrylic acid polymer.

2. The pharmaceutical composition according to claim 1, wherein said polypeptide is an endolysin having a killing activity against Gardnerella.

3. The pharmaceutical composition according to claim 2, wherein said endolysin comprises or consists of
(i) a N-terminal catalytic domain, or a functional variant thereof;
(ii) a C-terminal cell-wall binding region, or a functional variant thereof, wherein the C-terminal cell-wall binding region comprises or consists of at least one cell-wall binding domain; and
(iii) optionally a linker region between the N-terminal catalytic domain and the C-terminal cell-wall binding region.

4. The pharmaceutical composition according to any one of the preceding claims, wherein said polypeptide is a polypeptide having at least 80% sequence identity with the amino acid sequence as provided in SEQ ID NO: 1 or SEQ ID NO:37 and having a killing activity against Gardnerella.

5. The pharmaceutical composition according to any one of the preceding claims, wherein said polypeptide is a recombinant endolysin comprising the amino acid sequence provided in SEQ ID NO: 1 or SEQ ID NO: 37.

6. The pharmaceutical composition according to any one of the preceding claims, wherein said polypeptide has a killing activity against Gardnerella vaginalis sensu stricto, Gardnerella leopoldii, Gardnerella piotii and/or Gardnerella swidsinskii, or any other species in the genus Gardnerella.

7. The pharmaceutical composition according to any one of the preceding claims, wherein said polypeptide has a killing activity against Lactobacillus iners.

8. The pharmaceutical composition according to any one of the preceding claims, wherein said polypeptide has no killing activity against Lactobacilli crispatus, Lactobacilli gasseri, and/or Lactobacilli jensenii.

9. The pharmaceutical composition according to any one of the preceding claims, wherein said excipient is present at a concentration of about 0.01% and 10%, preferably about 0.1% to 1%, more preferably about 0.2% to 0.5%, most preferably about 0.25% or about 0.35%.

10. The pharmaceutical composition according to any one of the preceding claims, wherein the polyacrylic acid polymer is a crosslinked polyacrylic acid polymer or a carbomer.

11. The pharmaceutical composition according to any one of the preceding claims, wherein the combination of said active agent with said excipient increases the pharmaceutical activity of said active agent.

12. The pharmaceutical composition according to any one of the preceding claims, wherein the composition further comprises a salt.

13. The pharmaceutical composition according to claim 12, wherein the salt is sodium chloride (NaCl).

14. The pharmaceutical composition according to any one of the preceding claims, wherein said composition is suitable for intra-vaginal delivery of the active agent.

15. The pharmaceutical composition according to any one of the preceding claims, for use in treating a bacterial infection in a subject, preferably wherein the bacterial infection is bacterial vaginosis.

16. The pharmaceutical composition for use according to claim 15, wherein the subject previously failed a treatment with antibiotics and/or wherein the infective bacteria are resistant to a treatment with antibiotics.

17. The pharmaceutical composition for use according to claim 16, wherein said antibiotics are nitroimidazoles and/or Clindamycin.

18. The pharmaceutical composition for use according to claim 16 or 17, wherein the antibiotics are Metronidazole, Tinidazole, Secnidazole, Clindamycin or any combination thereof.

19. The pharmaceutical composition for use according to any one of claims 15 to 18, wherein said subject suffers from recurrent bacterial vaginosis, preferably wherein said subject had two or more episodes of BV in 6 months or had three or more episodes of BV in 12 months.

20. The pharmaceutical composition for use according to any one of claims 15 to 19, which is to be administered locally into the vagina of a female subject and/or into or on the glans penis, prepuce or urethral entry of a male subject.

21. Use of the pharmaceutical composition according to any one of claims 1 to 14 for the manufacture of a medicament for the treatment or the prevention of a bacterial infection, preferably bacterial vaginosis.

22. A method of treating or preventing a bacterial infection, preferably bacterial vaginosis, in a subject in need thereof comprising administering to said subject a therapeutically effective amount of the pharmaceutical composition according to any one of claims 1 to 14.
